(19) United States
(12) Patent Application Publication
(10) Pub. No.: US 2023/0165928 A1
(43) Pub. Date: Jun. 1, 2023

(54) COMPOSITIONS AND METHODS COMPRISING STABLE REDUCED GLUTATHIONE

(71) Applicants: Danny C. Purser, Provo, UT (US); Stephen N. PITCHER, Alpine, UT (US); Blue Sky Innovation, LLC, Pleasant Grove, UT (US)

(72) Inventors: Danny C. Purser, Provo, UT (US); Stephen N. Pitcher, Alpine, UT (US)

(73) Assignee: Blue Sky Innovation, LLC, Pleasant Grove, UT (US)

(21) Appl. No.: 17/922,452

(22) PCT Filed: Apr. 30, 2021
(86) PCT No.: PCT/US2021/030305

§ 371 Date: Oct. 31, 2022

Related U.S. Application Data
(60) Provisional application No. 63/018,419, filed on Apr. 30, 2020.

(51) Int. Cl. A61K 38/06; A61K 45/06; A61K 9/00; A61K 47/10

(52) U.S. Cl. CPC A61K 38/063; A61K 9/0014; A61K 45/06; A61K 47/10

(57) ABSTRACT

The present disclosure is drawn to a stable reduced glutathione composition. The stable reduced glutathione compositions can comprise an amount of reduced glutathione (rGSH) and a substantially a-D-glucopyranoside-free stabilizing carrier. A stable reduced glutathione delivery system can comprise: a container configured to minimize an amount of oxygen in the container; and a combination of reduced glutathione (rGSH) and a substantially a-D-glucopyranoside-free stabilizing carrier within the container. An oral dosage form for administration of a stable reduced glutathione (rGSH) can comprise: an amount of reduced glutathione (rGSH) and a substantially a-D-glucopyranoside-free stabilizing carrier which is suitable for oral administration.

### PRIORITY DATA

This application is a 371 U.S. Nationalization of International Pat. Cooperation Treaty Pat. Application No. PCT/US2021/030305, filed Apr. 30, 2021, which claims the benefit of U.S. Provisional Application Serial No. 63/018,419, filed Apr. 30, 2020, each of which is incorporated herein by reference in their entirety.

### TECHNOLOGY FIELD

The present disclosure relates to pharmaceutical compositions and dosage forms containing glutathione as well as associated methods. Accordingly, this disclosure involves the fields of chemistry, pharmaceutical sciences, medicine and other health sciences.

### BACKGROUND

Glutathione is a tripeptide comprised of three amino acids (cysteine, glutamic acid, and glycine) that can prevent damage to cellular components caused by reactive oxygen species (ROS) such as free radicals. Glutathione can exist in a reduced state (rGSH) and an oxidized state (GSSG). In healthy cells, more than 90% of the glutathione can be in the reduced state. Reduced glutathione plays various roles including in antioxidation, in detoxification, in thiol-exchange reactions, in metabolism, as an enzyme cofactor, and in leukotriene synthesis.

Bioavailability of exogenously administered rGSH can be poor because of reactions with reactive oxygen species during product storage and administration. Accordingly, in view of the potential therapeutic benefits offered by rGSH, stabile compositions and methods of administration that place a maximum amount of rGSH at target treatment situs continue to be sought.

### SUMMARY OF THE DISCLOSURE

The present disclosure is drawn to pharmaceutical compositions and dosage forms containing stable reduced glutathione (rGSH) and related methods. In one embodiment, a stable reduced glutathione composition is provided. The composition can comprise a combination of deoxygenated water and rGSH. The composition can be formulated such that more than about 80% of the glutathione remains in a reduced form after a period of about 365 days. In one aspect, the composition can be formulated such that more than about 80% of the glutathione remains in a reduced form after a period of about 365 days when placed in an airtight container. Additionally, the rGSH can be present in the composition at a concentration of from about 5 wt% to about 20 wt%. Further, the deoxygenated water can have less than about 5 mg/L of oxygen.

In another embodiment, a stable reduced glutathione composition is provided that includes an effective (e.g. therapeutically effective) amount of rGSH and a substantially α-D-glucopyranoside-free stabilizing carrier (e.g. the carrier is substantially free of α-D-glucopyranoside in either monomeric or polymeric form). Additionally, the stabilizing carrier comprise water or deoxygenated water. In some embodiments, the deoxygenated water can have less than about 5 mg/L of oxygen. Further, the rGSH can be present in the composition at a concentration of from about 5 wt% to about 20 wt%.

In an additional embodiment, a stable rGSH delivery system is provided. The stable rGSH delivery system can include a composition as recited herein and a container configured to minimize an amount of oxygen therein. The stable rGSH delivery system can include a combination of rGSH and a substantially α-D-glucopyranoside-free stabilizing carrier within the container. The container can be further configured to minimize the amount of oxygen in a headspace of the container and/or to minimize the entry of oxygen into the container.

In still a further embodiment, a method of treating a human subject responsive to treatment with rGSH is provided. In one embodiment, such a method can include administering a therapeutically effective amount of a composition or dosage form as recited herein to a subject.

In a yet further embodiment, a method of manufacturing a stabilized rGSH composition or dosage form is provided. Such a method can comprise combining a rGSH with deoxygenated water in a substantially inert environment to form an rGSH solution. The method can additionally comprise minimizing exposure of the rGSH solution to oxygen. Further, the method can comprise minimizing an amount of oxygen in a headspace of a container containing the rGSH solution. In some embodiments, such a method can comprise combining the reduced glutathione solution with a stabilizing agent.

### BRIEF DESCRIPTION OF THE DRAWINGS

For a fuller understanding of the nature and advantage of the present disclosure, reference is being made to the following detailed description of preferred embodiments and in connection with the accompanying drawings, in which:

FIG. 1 depicts a method of manufacturing a stabilized reduced glutathione composition in accordance with an example.

### DETAILED DESCRIPTION

Before disclosure embodiments are described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular examples or embodiments only and is not intended to be limiting. The same reference numerals in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of compositions, dosage forms, treatments, etc., to provide a thorough understanding of various disclosure embodiments. One skilled in the relevant art will recognize, however, that such detailed embodiments do not limit the overall inventive concepts articulated herein, but are merely representative thereof.

It has been discovered that rGSH and a substantially α-D-glucopyranoside-free stabilizing carrier is suitable to provide sustained, safe and effective increased rGSH levels relative to baseline levels (e.g. endogenous baseline levels) through effective and pragmatic dosing regimens (dose, dosing frequency, dosage units). Accordingly, it has been found that a dose of rGSH and a substantially α-D-glucopyranoside-free stabilizing carrier can achieve and maintain physiologically advantageous sustained rGSH levels in a subject. Moreover, such compositions/dosages can result in increased baseline levels of rGSH for a majority of a subject (e.g. patient) population when administered to a group.

It has been further discovered as mentioned in inventor’s U.S. Pat. No. 10,272,130, which is incorporated herein by reference, that a combination of deoxygenated water and rGSH is a suitable composition for providing therapeutically effective, sustained, and safe rGSH levels relative to a subject’s endogenous baseline levels through effective and pragmatic dosing regimens (dose, dosing frequency, dosage units).

In addition, is has been further discovered that combining rGSH with deoxygenated water in a substantially inert environment to form an rGSH solution and minimizing exposure of the rGSH solution to oxygen is suitable to provide stable rGSH for manufacturing and storage. It has been further discovered that a container configured to minimize an amount of oxygen in the container and a combination of reduced glutathione (rGSH) and a substantially α-D-glucopyranoside-free stabilizing carrier within the container is suitable for delivery of stable rGSH.

Contrary to expectations based on teachings in the art, it has been found that a composition comprising rGSH and a substantially α-D-glucopyranoside-free stabilizing carrier is effective in providing stable rGSH. Such a composition or dosage form is capable of maintaining rGSH in its reduced form over time during storage and to the point of administration to a subject, thus maximizing the amount of rGSH received by the subject and minimizing the amount of GSSG.

It should be noted that, the singular forms “a,” “an,” and, “the” include plural referents unless the context clearly dictates otherwise. Thus, for example, reference to “an excipient” includes reference to one or more of such excipients, and reference to “the carrier” includes reference to one or more of such carriers.

As is known in the art, the term “reduced glutathione” or “rGSH” generally refers to a compound having the structure:

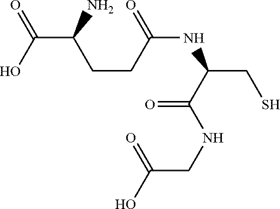 [SMILES: N[C@@H](CCC(=O)N[C@@H](CS)C(=O)NCC(=O)O)C(=O)O]

As used herein, the terms “oxidized glutathione” or “GSSG” or “glutathione disulfide” can be used interchangeably and refer to a compound having the structure:

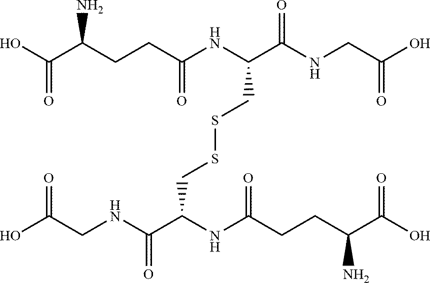 [SMILES: N[C@@H](CCC(=O)N[C@@H](CSSC[C@H](NC(=O)CC[C@H](N)C(=O)O)C(=O)NCC(=O)O)C(=O)NCC(=O)O)C(=O)O]

As used herein, the terms “treat,” “treatment,” or “treating” refers to administration of a therapeutic agent to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, the terms “therapeutic agent,” “active agent,” and the like can be used interchangeably and refer to agent that can have a beneficial or positive effect on a subject when administered to the subject in an appropriate or effective amount. In one aspect, the therapeutic or active agent can be an rGSH compound. The terms “additional active agent,” “supplemental active agent,” “secondary active agent,” and the like can be used interchangeably and refer to a compound, molecule, or material other than rGSH that has physiologic activity when administered to a subject in an effective amount.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients. Furthermore, the term “dosage form” can include one or more formulation(s) or composition(s) provided in a format for administration to a subject. For example, an “oral dosage form” can be suitable for administration to a subject’s mouth. A “topical dosage form” can be suitable for administration to a subject’s skin by rubbing, etc.

As used herein, the term “stabilizing agent” refers to a compound, molecule, or substance that has a stabilizing effect on rGSH or an additional active agent and increases the stability (e.g. reduces the degradation of rGSH into GSSH) while in a composition or dosage form.

As used herein, the term “fatty acid” refers to unionized carboxylic acids with a long aliphatic tail (chain), either saturated or unsaturated, conjugated or non-conjugated.

As used herein, the term “soluble” is a measure or characteristic of a substance or agent with regards to its ability to dissolve in a given solvent. The solubility of rGSH in a particular component of the composition refers to the amount of the rGSH dissolved to form a visibly clear solution at a specified temperature such as about 25° C. or about 37° C.

As used herein, the term “lipophilic,” refers to compounds that are not freely soluble in water; and the term “lipophilic surfactant” refers to surfactants that have HLB values of about 10 or less. Conversely, the term “hydrophilic” refers to compounds that are soluble in water; and the term “hydrophilic surfactant” refers to surfactants that have HLB values of more than about 10.

As used herein, the term “capsule fill” refers to the composition disposed in a capsule dosage form.

As used herein, a “subject” refers to an animal. In one aspect the animal may be a mammal. In another aspect, the mammal may be a human.

As used herein, a “responder” is a subject who responds positively to rGSH treatment or therapy. “Responder analysis” is the assessment of the effectiveness of rGSH therapy in a group of subjects deemed to get benefits of rGSH therapy.

As used herein, “group” or “group of subjects” refers to a collection of at least 3 human subjects who receive and respond to administration of the compositions disclosed herein, namely rGSH compositions. In one aspect, the group can include at least 20 or at least 50 subjects. In another aspect, the group can include at least 100 subjects.

The term “oral administration” represents any method of administration in which an active agent can be administered by swallowing, chewing, or sucking of the dosage form. Oral administration can be intended for enteral delivery of an active agent or transmucosal delivery of the active agent. In some embodiments, the composition of the current disclosure can be admixed with food or drink prior to being orally consumed.

As used herein, a “dosing regimen” or “regimen” such as an “initial dosing regimen” or “starting dose” or a “maintenance dosing regimen” refers to how, when, how much, and for how long a dose of the compositions of the present disclosure can be administered to a subject. For example, an initial or starting dose regimen for a subject may provide for a total daily dose of 600 mg administered in two divided doses at least 12 hours apart (e.g. once with breakfast and once with dinner) with meals repeated daily for 30 days.

As used herein, “daily dose” refers to the amount of active agent (e.g. rGSH) administered to a subject over a 24 hour period of time. The daily dose can be administered two or more administrations during the 24 hour period. In one embodiment, the daily dose provides for two administrations in a 24 hour period. With this in mind, an “initial dose” or initial daily dose” refers to a dose administered during the initial regimen or period of a dosing regimen.

As used herein, “non-liquid” when used to refer to the state of a composition disclosed herein refers to the physical state of the composition as being a semi-solid or solid.

As used herein, “solid” and “semi-solid” refers to the physical state of a composition that supports its own weight at standard temperature and pressure and has adequate viscosity or structure to not freely flow. Semi-solid materials may conform to the shape of a container under applied pressure.

As used herein, “titration” or “dose titration” or “dose adjustment” are used interchangeably and refer to an increase or decrease of the total daily dose of rGSH administered to a subject, typically based on the response of the subject to the exogenous administered rGSH. The dose can be increased or decreased based on the measurement of serum rGSH concentration after a steady state has been achieved.

As used herein, “steady state” refers to the achievement of stable serum total rGSH levels upon a continuous dosing regimen (e.g. once daily, twice daily etc.) of the administered rGSH at a given dose, after at least 7 consecutive days (typically achieved after at least 15 days), following the start of the dosing regimen. Unless otherwise stated, steady states values set forth herein refer to steady states achieved after a final dose titration (i.e., no additional titrations are required), including situations where no dose titration is required. Similarly, as used herein, the “steady state serum concentration (Cₛₛ, Css)” or “mean steady state serum concentration (mean Cₛₛ)” of rGSH refers to the achievement of a stable serum total rGSH concentration in a subject or group of subjects, respectively, in response to a continuous dosing regimen (e.g. once daily, twice daily etc.) of the administered rGSH at a given dose, after at least 7 days (typically achieved after at least 15 days), following the start of the dosing regimen. It should be further noted that the when a dose adjustment (increase or decrease in total daily dose of rGSH administered) is made as part of the dose-titration during the treatment, the mean Cₛₛ is achieved at least about 7 days after the initiation of the change in the dose administered.

As used herein, the terms “release” and “release rate” are used interchangeably to refer to the discharge or liberation of a substance, including without limitation a drug, from the dosage form into a surrounding environment such as an aqueous medium either in vitro or in vivo.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, the term “delayed release” refers to the release into an aqueous solution of the rGSH from the composition or oral dosage form in a time delayed manner attributed either to the inherent nature of the composition or to a coating which may surround the composition or the oral dosage form. In one embodiment, the delayed release is such that about 20% or less of the rGSH is released within the first 15 minutes after the composition is contacted by the aqueous solution.

The terms “serum rGSH levels,” “plasma rGSH concentration,” “rGSH concentration in the blood,” and “serum rGSH concentration,” are used interchangeably and refer to the “total” rGSH concentration which is the sum of the bioavailable rGSH including free and bound (if any) rGSH concentrations. Unless otherwise specified, these values are “observed rGSH concentrations without adjusting or correcting for the baseline serum rGSH levels in the subject(s). As with any bio-analytical measure, for increased consistency the method employed to measure initial serum rGSH levels should be consistent with the method used to monitor and re-measure serum rGSH levels during clinical testing and rGSH therapy for a subject. Unless otherwise stated, “rGSH concentration” refers to serum total rGSH concentration.

As used herein, the average serum rGSH concentration can be determined using methods and practices known in the art. For example, the average baseline plasma rGSH concentration of a subject can be the arithmetic mean of the total plasma rGSH concentrations determined on at least two consecutive time points that are reasonably spaced from each other, for example from about 1 hour to about 168 hours apart. In a particular case, the plasma rGSH concentration can be determined on at least two consecutive times that are about 12 hours to about 48 hours apart. In another particular method, the plasma rGSH concentration of the subject can be determined at a time between about 5 o’clock and about 11 o’clock in the morning. Further, the plasma rGSH concentration can be determined by standard analytical procedures and methods available in the art, such as for example, automated or manual immunoassay methods, liquid chromatography or liquid chromatography- tandem mass spectrometry (LC-MSMS) etc.

As used herein, the term AUCₜ₁₋ₜ₂ is the area under the curve of a plasma-versus-time graph determined for the analyte from the time “t1 to time t2”. Wherein t1 and t2 are times (in hours) post dosing. For Example, t1 could be 1 hour and t2 could be 2 hours.

As used herein, the term “C_{avg},” “Cₐᵥₑ,” or “C-average” are used interchangeably, and is determined as the AUCₜ₁₋ₜ₂ mean AUC divided by the time period (|t1-t2|). For example, C_{avg} ₜ₀₋ₜ₈ is the average plasma concentration over a period of 8 hours from t1=0 to t2=8 hours) post-dosing determined by dividing the AUC t₀₋ₜ₈ value by 8. Similarly, C_{avg t0-t12} is the average plasma concentration over a period of 12 hours post-dosing determined by dividing the AUCₜ₀₋ₜ₁₂ value by 12 (t1=0-t2=12). Similarly, C_{avg} ₜ₁₂₋ₜ₂₄ is the average plasma concentration over a period of 12 hours post-dosing determined by dividing the AUCt₁₂₋ₜ₂₄ value by 12 (t1=12-t2=24);C_{avg-t24} is the average plasma concentration over a period of 24 hours post-dosing determined by dividing the AUCt₀₋ₜ₂₄ value by 24 (t1=0-t2=24), and so on. Unless otherwise stated, all C_{avg} values are considered to be C_{avg-t24} and unless otherwise stated, all the time values are expressed in hours (h). For example, the term C_{avg t0-t24} denotes C_{avg} from time zero (0) to 24 hours post dosing.

As used herein, “Ct” refers to the serum concentration of rGSH at time “t” prior to or after administration of the dosage of the current disclosure. The time “t” is generally in hours, unless otherwise specified. For example, a Cₜ of “C₍₋₂ ₜₒ ₀₎refers to serum rGSH concentration measured in sample collected between the time of about 2 hours before and just immediately prior to dosage administration to the subject tested. Similarly, Cₜ of “C₍₂ ₜₒ ₄₎” refers to serum rGSH concentration measured in sample collected between the time of about 2 hours and 4 hours after administration of a dosage to the subject tested.

As used herein “single unit” when used to describe dosing of a subject refers to the dosage form being a single dosage form, e.g. a single tablet, capsule, pump or squirt of gel or solution, etc. In contrast, “multiple unit” when used to describe dosing of a subject refers to the dosage including two or more dosage forms, e.g. 2 tablets, 3 capsules, 2-4 pumps or squirts, etc. It is noteworthy that multiple unit dosage forms generally will be the same type of dosage forms (i.e. tablet or capsule) but are not required to be the same dosage form type.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” “maximized,” “minimized,” and the like refer to a property of a device, component, composition, or activity that is measurably different from other devices, components, compositions or activities that are in a surrounding or adjacent area, that are similarly situated, that are in a single device or composition or in multiple comparable devices or compositions, that are in a group or class, that are in multiple groups or classes, or as compared to the known state of the art. For example, a composition that has “increased” stability of rGSH keeps a higher amount of its glutathione content in a reduced form (rGSH) rather than converting to an oxidized form (GSSH) for the same or greater amount of time as compared to a similar composition which does not achieve such results.

The term “coupled,” as used herein, is defined as directly or indirectly connected in a chemical, mechanical, electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

With the above described background in mind, the inventors have identified a need for an efficient and patient-friendly means to help increase and/or restore rGSH levels in a subject. For example, compositions, dosage forms and related methods have been discovered which are capable of increasing serum rGSH levels in most patients for longer periods of time post dosing in large percentage patients. Further, the compositions, dosage forms and related methods disclosed herein are able to accomplish these desirable results while still providing patient-friendly regimens - such as, a practical rGSH equivalent daily dose, topical and transmucosal dosage forms, and fewer number of dosage units per administration.

It has been discovered that rGSH and a substantially α-D-glucopyranoside-free stabilizing carrier is suitable to provide sustained, safe and effective increased rGSH levels relative to baseline levels through effective and pragmatic dosing regimens (dose, dosing frequency, dosage units). Accordingly, it has been found that a dose of rGSH and a substantially α-D-glucopyranoside-free stabilizing carrier can offer adequate bioactivity and bioavailability as compared to rGSH, and maintain sustained rGSH levels in a patient. Moreover, the compositions/dosage forms of rGSH and a substantially α-D-glucopyranoside-free stabilizing carrier at their daily dose when administered to a group of subjects, result in increased baseline levels of rGSH for the majority of subjects.

It has been further discovered that a combination of deoxygenated water and rGSH is suitable to provide sustained, safe and effective increased rGSH levels relative to baseline levels through effective and pragmatic dosing regimens (dose, dosing frequency, dosage units). Accordingly, it has been found that a dose of a combination of deoxygenated water and rGSH can offer increased bioactivity and bioavailability as compared to baseline levels, and maintain increased rGSH levels in a patient. Moreover, the compositions/dosage forms of a combination of deoxygenated water and rGSH at their daily dose when administered to a group of subjects, results in increased baseline levels of rGSH for the majority of subjects.

In addition, is has been further discovered that combining rGSH with deoxygenated water in a substantially inert environment to form an rGSH solution and minimizing exposure of the rGSH solution to oxygen is suitable to provide stable rGSH for manufacturing and storage. It has been further discovered that a container configured to minimize an amount of oxygen in the container and a combination of reduced glutathione (rGSH) and a substantially α-D-glucopyranoside-free stabilizing carrier within the container is suitable for delivery of stable rGSH.

With this in mind, in one embodiment of the present disclosure, a stable rGSH composition is provided. In one embodiment, such a composition can comprise a combination of deoxygenated water and rGSH, with more than about 80 % of the glutathione remaining in a reduced form after a period of about 30 days when stored at ambient pressure and temperature. In one aspect, the rGSH can be present in the composition at a concentration of from about 5 wt% to about 20 wt%. The deoxygenated water can have less than about 5 mg/L of oxygen. In some aspects, the amount of glutathione remaining in the reduced form after the period of 365 days can be greater than 80% when stored at ambient pressure and temperature. In one aspect, the amount of glutathione remaining in the reduced form after the period of 365 days can be greater than 80% when stored at ambient pressure and temperature when placed in an airtight container.

In another embodiment, stable reduced glutathione compositions can comprise an amount (e.g. an effective or therapeutically effective amount) of rGSH and a substantially α-D-glucopyranoside-free stabilizing carrier. Moreover, such a composition can be further substantially free of macro-cyclic glucose subunits, oligosaccharides, glucopyranoside units, or glycosidic bonds. Such compositions can have at least the above-recited stability performance properties or better (e.g. greater than 85% of glutathione remaining as rGSH after from 30 days to 365 days when stored at ambient pressure and temperature or under other conditions). Additionally, in some embodiments, the stabilizing carrier can include deoxygenated water as recited herein.

In some embodiments, the compositions can further comprise an additional active agent. The additional active agent can be a member selected from the group consisting of: an antioxidant, N-acetyl-cysteine, hydroxytyrosol (HXT), superoxide dismutase (SOD), catalase, an anti-infective agent, an antibiotic, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

In further embodiments, the compositions can further comprise a support agent selected from the group consisting of: a stabilizing agent, a preservative, an adjuvant, an emollient, a carrier, sunflower oil, natural lemon essential oil, the like, and combinations thereof. In some embodiments, the support agent can be a stabilizing agent. Such a stabilizing agent can include an oxygen scavenger, a lipid, a synthetic polymer, a natural non-α-D-glucopyranoside polymer, a protein, an amino acid, a co-polymer, an inert gas, or a combination thereof.

In addition, the stabilizing agent can be a lipid selected from the group consisting essentially of: phospholipids, glycolipids, cholesterol, triglycerides, fatty acids, fatty acid glycerides, surfactants, or a combination thereof. The lipid can form a liposome, an inverted micelle, or a combination thereof. The composition can be formulated as one of a solution, a suspension, an emulsion, a gel, a hydrogel, a thermo-responsive gel, a cream, an ointment, a paste, an adhesive, an erodible matrix, a liquid reservoir, a patch, a powder, a compressed powder, or a combination thereof.

In some embodiments, the composition can be formatted in a dosage form comprising a transdermal dosage form, a parenteral dosage form, a topical dosage form, an oral dosage form, a nebulizer dosage form, a transmucosal dosage form, and combinations thereof. Carriers, including various excipients, emollients, adjuvants, and other additives can be used in order to provide a specific dosage form. In some embodiments, a therapeutically effective amount of the composition can result in a serum level of rGSH in a subject that is between 5% and 50% greater than a baseline level for the subject during a period of time of up to 24 hours after administration.

In another embodiment of the present disclosure, a method of manufacturing an rGSH composition is provided. Such a method can include combining rGSH with a stabilizing carrier as recited herein (including a carrier that utilizes deoxygenated water) in a substantially inert environment to form an rGSH solution. Such a method can include minimizing exposure of the rGSH solution to oxygen, which can further include minimizing an amount of oxygen in a headspace of a container containing the rGSH solution.

Furthermore, such a method can include adding an additional active agent such as vitamin C, vitamin E, an antioxidant, N-acetyl-cysteine, hydroxytyrosol (HXT), superoxide dismutase (SOD), catalase, an anti-infective agent, an antibiotic, an anti-tumor agent, an anti-inflammatory agent, an analgesic, an anti-rheumatic agent, a supplementary growth factor, a supplementary cytokine, an amino acid, a protein, a vaccine, a hormone, a supplementary vitamin, oleuropein, and combinations thereof.

In another embodiment of the present disclosure, a stable rGSH delivery system is provided. The system can include a container configured to minimize an amount of oxygen in the container. The system can include a combination of rGSH and a substantially α-D-glucopyranoside-free stabilizing carrier within the container.

In some embodiments, the container can be further configured to minimize the amount of oxygen in a headspace of the container. One mechanism of minimizing oxygen is to make the container air-tight. Another is to select materials for the container that are inert and won’t leach oxygen into the rGSH composition. Further, the headspace of the container can be minimized. In one embodiment, the headspace can be less than about 4% of the container volume. The amount of oxygen in the container can be less than about 5% of the container volume. The container can include (e.g. in the headspace) an inert compound including one or more of: nitrogen gas, helium gas, neon gas, argon gas, krypton gas, xenon gas, radon gas, a non-reactive compound, a non-oxidizing compound, the like, or combinations thereof. The container can be a bag-on-valve container, a bag-in-bag container, a piston can container, or a can-in-can container.

In another embodiment of the present disclosure, methods of treating a human subject responsive to treatment with reduced glutathione is provided. Such a method can include administering a therapeutically effective amount of the composition as provided herein to the subject.

In yet other embodiments, specific dosage forms for delivering an effective amount of rGSH and/or providing rGSH therapy are provided. In some aspects, such dosage forms can include an effective (e.g. therapeutically effective) amount of rGSH in combination with a stabilizing carrier that is effective in maximizing the amount of glutathione that remains in a reduced form. In some aspects, such a stabilizing carrier can include water and optionally, one or more stabilizing agents as recited herein. In some embodiments, the carrier can comprise deoxygenated water. In other embodiments, the carrier can be substantially free of one or more of α-D-glucopyranoside units. In some embodiments, the dosage forms can have the same or similar basic chemical and/or physical properties as discussed herein with respect to the compositions, namely, materials, ingredients, components, amounts, concentrations, etc. while being specifically prepared for specific types or routes of administration.

More specifically, the discussion herein relating to compositional components that can be used in the pharmaceutical compositions is also equally applicable to the dosage form embodiments and related methods disclosed herein unless expressly stated to the contrary. It is also noteworthy that the discussion relating to compositional components that can be used in the pharmaceutical compositions is also equally applicable to the dosage form embodiments and related methods disclosed herein unless expressly stated to the contrary. Thus, for example, teachings regarding the use of stabilizing agents for use in the pharmaceutical compositions disclosed herein are also equally applicable to the dosage forms and related methods described herein and vice versa.

In one embodiment of the present disclosure, an oral dosage form for administration of an rGSH is provided. The oral dosage form can comprise an amount of rGSH and a substantially α-D-glucopyranoside-free stabilizing carrier which is suitable for oral administration. The carrier can comprise one or more of: binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof. The rGSH can be present in an amount of about 50 mg to about 1500 mg. The oral dosage form can be a solution, a gel, a capsule or a tablet. The oral dosage form can be either immediate release or controlled (e.g. delayed, sustained, pulsatile, etc.) release.

When compositions are formulated as oral dosage forms, such as a solution, a capsule or tablet dosage form, the dosage form can include about 50 mg to about 1500 mg of the rGSH. In a specific embodiment, when the oral dosage compositions are formulated as oral dosage forms, such as a capsule, the dosage form can include about 200 mg to about 1200 mg of the rGSH. In one embodiment, the daily dose of the rGSH can be about 300 mg to about 800 mg. In another embodiment, the daily dose of the rGSH can be from about 400 mg to about 600 mg. In one embodiment, the daily dose of the rGSH can be about 300 mg to about 500 mg. In another embodiment, the daily dose of the rGSH can be from about 600 mg to about 800 mg. In one embodiment, the rGSH can comprise about 0.5% to 50% by weight of the oral pharmaceutical composition or capsule dosage form. In another embodiment, the rGSH can comprise about 5% to 50%, or 10-50% or 15-50% or 10-35% or 10-30% or 10-25% or 15-25% or 15-30% or 20-30% or 20-25%, or more specifically, about 35%, about 30%, about 25%, about 20%, about 18%, about 16%, about 15%, about 12%, or about 10% by weight composition, of the oral pharmaceutical composition or dosage form (e.g. capsule or tablet).

In another embodiment of the present disclosure, a topical dosage form for administration of an rGSH is provided. The topical dosage form can include an amount of rGSH and a substantially α-D-glucopyranoside-free stabilizing carrier which is suitable for topical administration. The carrier can be one or more of a gel, a cream, an ointment, a liniment, a paste, or a lotion. The rGSH can be present in an amount of about 50 mg to about 1500 mg.

In yet another embodiment of the present disclosure, a transdermal dosage form for administration of an rGSH is provided. The transdermal dosage form can include an amount of rGSH and a substantially α-D-glucopyranoside-free stabilizing carrier which is suitable for transdermal administration. The carrier can include one or more of water, a polymer having surfactant properties, a polymer having thickening properties, or a solvent. The rGSH can be present in an amount of about 50 mg to about 1500 mg. The transdermal dosage form can further comprise a penetration enhancer.

In another embodiment of the present disclosure, a parenteral dosage form for administration of an rGSH is provided. The parenteral dosage form can include an amount of rGSH and a substantially α-D-glucopyranoside-free stabilizing carrier which is suitable for parenteral administration. The carrier can comprise one or more of water, a tonicity agent, a buffering agent, a preservative, or a combination thereof. The rGSH can be present in an amount of about 1 mg to about 1500 mg. The parenteral dosage form can be suitable for administration via intradermal injection, subcutaneous injection, intramuscular injection, intraperitoneal injection, or intravenous injection.

In a further embodiment of the present disclosure, a transmucosal dosage form for administration of an rGSH is provided. The transmucosal dosage form can include an amount of rGSH and a substantially α-D-glucopyranoside-free stabilizing carrier which is suitable for transmucosal administration. The rGSH can be present in an amount of about 50 mg to about 1500 mg. In some embodiments, the transmucosal dosage form can further include an adhesive layer and/or a bio-erodible matrix. The transmucosal dosage form can be suitable for nasal, vaginal, or rectal administration.

In addition to the rGSH, the pharmaceutical compositions and dosage forms can further include at least one additional pharmaceutically active agent or can be formulated to be co-administered with other active agents in order to treat a target condition. Non-limiting examples of additional active agents that can be included with or co-administered with the pharmaceutical composition or dosage form include: an antioxidant, N-acetyl-cysteine, hydroxytyrosol (HXT), superoxide dismutase (SOD), catalase, an anti-infective agent, an antibiotic, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof. Such compounds could be co-administered with the compositions and dosage forms of the present disclosure in order to provide improved clinical outcomes.

Antioxidants can be included in the present compositions and dosage forms for their effect as an additional active agent or in order to help in stabilizing the rGSH. Various antioxidants can be used including, but not limited to: N-acetyl-cysteine, hydroxytyrosol (HXT), superoxide dismutase (SOD), catalase, Vitamin A, Vitamin C, Vitamin E, coenzyme Q10, managanese, iodide, melatonin, alpha-carotene, astaxanthin, beta-carotene, canthaxanthin, cryptoxanthin, lutein, lycopene, zeaxanthin, apigenin, luteolin, tangeritin, isorhamnetin, kaempferol, myricetin, proanthocyanidins, quercetin, eriodyctiol, hesperetin, naringenin, catechin, gallocatechin, epicatechin, epigallocatechin, theaflavin, thearubigins, daidzein, genistein, glycitein, resveratrol, pterostilbene, cyanidin, delphinidin, malvidin, pelargonidin, peonidin, petunidin, chicoric acid, cholorogenic acid, cinnamic acid, ellagic acid, ellagitannins, gallic acid, gallotannins, rosmarinic acid, slaicyclic acid, curcumin, flavonolignans, xanthones, eugenol, capsaicin, bilirubin, citric acid, oxalic acid, phytic acid, R-alpha-Lipoic acid, the like, and combinations thereof.

The additional active agent can include one or more anti-infective agents, namely an agent that can kill or prevent an infectious organism (e.g. a pathogen) from spreading. Thus, anti-infective agents can include antibacterial agents, antifungal agents, antiviral agents, antiprotozoan agents, the like, or combinations thereof. Non-limiting examples can include amebicides such as chloroquine, nitazoxanide, paromomycin, tinidazole, metronidazole, iodoquinole, or the like; aminoglycosides such as tobramycin, gentamicin, amikacin, kanamycin, neomycin, streptomycin, or the like; anthelmintics such as albendazole, ivermectin, praziquantel, pyrantel, mebendazole, miltefosine, niclosamide, piperazine, thiabendazole, or the like; antifungals such as itraconazole, posaconazole, ketoconazole, fluconazole, clotrimazole, isavuconazole, miconazole, voriconazole, echinocandins, terbinafine, griseofulvin, flucytosine, nystatin, amphotericin b, or the like; antimalarials such as chloroquine, quinine, hydroxychloroquine, mefloquine, primaquine, pyrimethamine, halofantrine, doxycycline, or the like; antituberculosis agents such as aminosalicylic acid, bedaquiline, isoniazid, ethambutol, pyrazinamide, ethionamide, rifampin, rifabutin, rifapentine, capreomycin, cycloserine, streptomycin, or the like; antivirals such as amantadine, rimantadine, ritonavir, cobicistat, peginterferon alfa-2a, peginterferon alfa 2b, maraviroc, raltegravir, dolutegravir, elvitegravir, sofosbuvir, enfuvirtide, fomivirsen, foscarnet, oseltamivir, zanamivir, peramivir, etravirine, efavirenz, nevirapine, delavirdine, rilpivirine, daclatasvir, adefovir, entecavir, telbivudine, didanosine, tenofovir, abacavir, lamivudine, zidovudine, stavudine, emtricitabine, zalcitabine, boceprevir, simeprevir, fosamprenavir, lopinavir, darunavir, telaprevir, ritonavir, tipranavir, atazanavir, nelfinavir, amprenavir, indinavir, saquinavir, ganciclovir, valacyclovir, famciclovir, acyclovir, valganciclovir, ribavirin, cidofovir, or the like; carbapenems such as doripenem, meropenem, cilastatin, ertapenem, or the like; cephalosporins such as avibactam, ceftolozane, ceftazidime, tazobactam, cefadroxil, cephalexin, cefazolin, ceftaroline, loracarbef, cefotetan, cefuroxime, cefprozil, cefaclor, cefoxitin, ceftibuten, cefotaxime, ceftriaxone, cefpodoxime, cefixime, cefdinir, defditoren, ceftazidime, ceftizoxime, cefepime, or the like; glycopeptide antibiotics such as vancomycin, dalbavancin, oritavancin, telavancin, or the like; glycocyclines such as tigecycline, or the like; leprostatics such as thalidomide, dapsone, clofazimine, or the like; lincomycin, or the like; clindamycin, or the like; ketolides such as telithromycin, or the like; macrolides such as azithromycin, fidaxomicin, erythromycin, clarithromycin, or the like; antibiotics such as aztreonam, daptomycin, chloramphenicol, colistimethate, fosfomycin, rifaximin, metronidazole, sulfamethoxazole, atovaquone, bacitracin, dalfopristin, erythromycin, furazolidone, pentamidine, polymyxin b, spectinomycin, trimetrexate, linezolid, tedizolid, penicillins (e.g. ampicillin, amoxicillin, carbenicillin, piperacillin, ticarcillin, nafcillin, dicloxacillin, cloxacillin, oxacillin, or the like), quinolones (e.g. lomefloxacin, norfloxacin, ofloxacin, gatifloxacin, moxifloxacin, ciprofloxacin, levofloxacin, gemifloxacin, cinoxacin, nalidixic acid, sparfloxacin, or the like), sulfonamides (e.g. sulfamethoxazole, sulfadiazine, sulfisoxazole, or the like), tetracyclines (e.g. tetracycline, demeclocycline, doxycycline, minocycline, or the like), or the like; urinary anti-infectives such as methenamine, methylene blue, fosfomycin, nitrofurantoin, trimethoprim, cinoxacin, nalidixic acid, oxytetracycline, or the like; hydrates thereof, acids thereof, bases thereof, salts thereof, or combinations of any of such anti-infective agents.

In some examples, the additional active agent can also include any suitable antitumor agent. Non-limiting examples of antitumor agents can include angiogenesis inhibitors such as angiostatin k1-3, angiostatin k1-5, DL-α-difluoromethylomithine, endostatin, fumagillin, genistein, minocycline, staurosporine, (+/-)-thalidomide, or the like; DNA intercalators such as bleomycin, carboplatin, carmustine, chlorambucil, cisplatin, cyclophosphamide, cis-diammineplatinum(II) dichloride, melphalan, mitoxantrone, oxaliplatin, or the like; DNA synthesis inhibitors such as (+/-)-amethopterin, 3-amino-1,2,4-benzotraizine-1,4-dioxide, aminopterin, cytosine β-D-arabinofuranoside, 5-fluoro-5′-deoxyuridine, 5-fluorouracil, ganciclovir, hydroxyurea, mitomycin C, or the like; transcriptioin regulators such as actinomycin D, daunorubicin, doxorubicin, homoharringtonine, idarubicin, or the like; enzyme inhibitors such as S(+)-camptothecin, curcumin, (-)-deguelin, 5,6-dichlorobenzimidazole 1-β-D-ribofuranoside, etoposide, formestane, fostriecin, hispidin, 2-imino-1-imidazolidineacetic acid, mevinolin, trichostatin A, tyrphostin AG 34, tyrphostin AG 879, or the like; gene regulation agents such as 5-aza-2′-deoxycytidine, 5-azacytidine, cholecalciferol, 4-hydroxytamoxifen, melatonin, mifepristone, raloxifene, vitamin A aldehyde, vitamin A acid, vitamin A, 9-cis-retinoic acid, 13-cis-retinoic acid, tamoxifen, troglitazone, or the like; microtubule inhibitors such as colchicine, docetaxel, dolastatin 15, etoposide, irinotecan, nocodazole, paclitaxel, podophyllotoxin, rhizoxin, vinblastine, vincristine, vindesine, vinorelbine, or the like; other antitumor agents such as 17-(allylamino)-17-demethoxygeldanamycin, 4-amino-1,8-naphthalimide, apigenin, brefeldin A, cimetidine, dichloromethylene-diphosphonic acid, leuprolide, luteinizing hormone-releasing hormone, pifithrin-α, rapamycin, sex hormone-binding globulin, thapsigargin, bikunin, ifosfamide, temozolomide, capecitabine, methotrexate, gemcitabine, pemetrexed, mitomycin, epirubicin, bevacizumab, cetuximab, gefitinib, imatinib, trastuzamab, denosumab, rituximab, sunitinib, zoledronate, abiraterone, anastrozole, bicalutamide, exemestane, goserelin, medroxyprogesterone, octreotide, tamoxifen, bendamustine, lomustine, procarbazine, streptozocin, fludarabine, raltitrexed, mitoxantrone, eribulin, topotecan, afatinib, aflibercept, BCG, crizotinib, dabrafenib, interferon, ipilimumab, lapatinib, nivolumab, panitumumab, pembrolizumab, pertuzumab, sorafenib, trastuzumab emtansine, temsirolimus, vemurafenib, ibandronic acid, pamidronate, bexarotene, buserelin, cyproterone, degarelix, folinic acid, fulvestrant, lanreotide, lenalidomide, letrozole, leuprorelin, megestrol, mesna, thalidomide, or the like; hydrates thereof, acids thereof, bases thereof, salts thereof, or combinations of any of such antitumor agents.

In some examples, the additional active agent can also include any suitable anti-inflammatory agent. Non-limiting examples of anti-inflammatory agents can include ibuprofen, naproxen, aspirin, diclofenac, celecoxib, sulindac, oxaprozin, piroxicam, indomethacin, meloxicam, fenoprofen, difunisal, etodolac, ketorolac, meclofenamate, nabumetone, salsalate, ketoprofen, tolmetin, flurbiprofen, mefenamic acid, famotidine, bromfenac, nepafenac, prednisone, cortisone, hydrocortisone, methylprednisolone, deflazacort, prednisolone, fludrocortisone, amcinonide, betamethasone diproprionate, clobetasol, clocortolone, dexamethasone, diflorasone, durasteride, flumethasone pivalate, flunisolide, fluocinolone acetonide, fluocinonide, fluorometholone, fluticasone propionate, flurandrenolide, hydroflumethiazide, the like, hydrates thereof, acids thereof, bases thereof, or salts thereof, or combinations thereof.

In some examples, the additional active agent can also include any suitable analgesic. Non-limiting examples of analgesics can include anti-inflammatory agents, such as those listed above, acetaminophen, codeine, dihydrocodeine, tramadol, meperidine, hydrocodone, oxycodone, morphine, fentanyl, hydromorphone, buprenorphine, methadone, diamorphine, pethidine, the like, hydrates thereof, acids thereof, bases thereof, or salts thereof, or combinations thereof.

In some examples, the additional active agent can also include any suitable anti-rheumatic agent. Non-limiting examples of anti-rheumatic agents can include methotrexate, sulfasalazine, chloroquine, hydroxychloroquine, leflunomide, azathioprine, cyclosporine, minocycline, abatacept, rituximab, tocilizumab, anakinra, adalimumab, etanercept, infliximab, cetolizumab, golimumab, D-penicillamine, auranofin, the like, hydrates thereof, acids thereof, bases thereof, or salts thereof, or combinations thereof.

In some examples, the additional active agent can also include any suitable growth factor. Non-limiting examples of supplementary growth factors can include platelet-derived growth factor (PDGF), epidermal growth factor (EGF), transforming growth factor- alpha (TGF-α), fibroblast growth factor (FGF), nerve growth factor (NGF), erythropoietin, transforming growth factor-beta (TGF-β), insulin-like growth factor-1 (IGF-1), insulin-like growth factor-2 (IGF-2), the like, or combinations thereof.

In some examples, the additional active agent can also include any suitable supplementary cytokine. Non-limiting examples of supplementary cytokines can include interleukins, lymphokines, monokines, interferons, colony stimulating factors, chemokines, the like, or combinations thereof.

In some examples, the additional active agent can also include any suitable amino acid. Non-limiting examples can include arginine, histidine, isoleucine, leucine, lysine, methionine, phenylalanine, tryptophan, valine, acetyl-L-carinitine arginate, alpha-aminoadipic acid, alpha-amino-N-butyric acid, beta-alanine, beta-amino-isobutyric acid, carnosine, citruline, gamma-amino butyric acid, hydroxyproline, 1-methylhistidine, 3-methylhistidine, N-acytyl-L-cysteine, ornithine, para-aminobenzoic acid, phosphoserine, phosphoethanolamine, taurine, the like, isomers thereof, hydrates thereof, salts thereof, acids thereof, bases thereof, or any combinations thereof.

In some examples, the additional active agent can also include any suitable protein. Non-limiting examples can include cytokines and/or growth factors, such as those listed above, as well as antibodies, Fc-fusion proteins, anticoagulants, blood factors, bone morphogenetic proteins, engineered protein scaffolds, enzymes, hormones, thrombolytics, the like, or combinations thereof.

In some examples, the additional active agent can also include a vaccine. Non-limiting examples of vaccines can include adenovirus vaccine, coxsackie B vaccine, cytomegalovirus vaccine, dengue vaccine, Eastern equine encephalitis vaccine, ebola vaccine, enterovirus vaccine, Epstein-barr vaccine, hepatitis A vaccine, hepatitis B vaccine, hepatitis C vaccine, hepatitis E vaccine, HIV vaccine, human papillomavirus vaccine, HTLV-1 T-lymphotrophic vaccine, influenza vaccine, Japanese encephalitis vaccine, Marburg vaccine, measles vaccine, mumps vaccine, norovirus vaccine, polio vaccine, rabies vaccine, respiratory syncytial virus (RSV) vaccine, rotavirus vaccine, rubella vaccine, severe acute respiratory syndrome (SARS) vaccine, varicella vaccine, smallpox vaccine, West Nile virus vaccine, yellow fever vaccine, anthrax vaccine, DPT vaccine, Q fever vaccine, Hib vaccine, tuberculosis vaccine, meningococcal vaccine, typhoid vaccine, pneumococcal vaccine, cholera vaccine, caries vaccine, ehrlichiosis vaccine, leprosy vaccine, lyme disease vaccine, staphylococcus aureus vaccine, streptococcus pyogenes vaccine, syphilis vaccine, tularemia vaccine, Yersinia pestis vaccine, the like, or combinations thereof.

In some examples, the additional active agent can also include a hormone. Non-limiting examples of hormones can include progestogens, androgens, estrogens, somatostatins, growth hormones, thyroid hormones, glucocorticoids, the like, or combinations thereof.

In some examples, the additional active agent can also include a vitamin. Non-limiting vitamins can include vitamin A, vitamin B1, vitamin B2, vitamin B6, vitamin B12, vitamin C, vitamin D, vitamin E, vitamin H, vitamin K, folic acid, the like, or combinations thereof.

In some examples, the additional active agent can also include phenolic compounds. Non-limiting phenolic compounds can include oleuropein, demethyl-oleuropein, ligstroside, and oleoside, oleuropein, tyrosol, hydroxytyrosol, caffeic acid, the like, and combinations thereof. In another example, the additional active agent can include oleuropein.

Further, in addition to rGSH, the pharmaceutical compositions and dosage forms can further include a support agent selected from the group consisting of: a stabilizing agent, a preservative, an adjuvant, an emollient, a carrier, sunflower oil, natural lemon essential oil, the like, and combinations thereof.

In another aspect, the support agent can be a stabilizing agent. In one embodiment, the stabilizing agent can comprise about 0.5% to 50% by weight of the pharmaceutical composition or dosage form. In another embodiment, the stabilizing agent can comprise about 5% to 50%, or 10-50% or 15-50% or 10-35% or 10-30% or 10-25% or 15-25% or 15-30% or 20-30% or 20-25%, or more specifically, about 35%, about 30%, about 25%, about 20%, about 18%, about 16%, about 15%, about 12%, about 10%, about 8%, about 6%, about 4%, or about 2% by weight composition, of the pharmaceutical composition or dosage form.

The stabilizing agent can include an oxygen scavenger, a synthetic polymer, a natural non-α-D-glucopyranoside polymer, a lipid, a protein, an amino acid, a co-polymer, an inert gas, or a combination thereof.

In some examples, the stabilizing agent can include an oxygen scavenger. Non-limiting examples of oxygen scavengers can include, but are not limited to, suitable combinations of: a transition metal catalyst (e.g., copper), ascorbic acid, glucose oxidase, catalase, polyunsaturated fatty-acids, soybean oil, sesame oil, cottonseed oil, squalene, fatty acids, polybutadiene, yeast, antioxidants, the like, and combinations thereof. In some examples, the stabilizing agent can include a synthetic polymer. In some examples, the stabilizing agent can include a natural non-α-D-glucopyranoside polymer.

In another aspect, the stabilizing agent can be a lipid selected from the group consisting essentially of: phospholipids, glycolipids, cholesterol, triglycerides, fatty acids, fatty acid glycerides, surfactants, or a combination thereof. In an example, a phospholipid can include two hydrophobic fatty acid tails and a hydrophilic head consisting of a phosphate group. In an example, a phospholipid can be a glycerol-based phospholipid including, but not limited to, plasmalogens, phosphatidates, phosphatidylcholines, or glycolipids.

In another example, the lipid can form a liposome, an inverted micelle, or a combination thereof. Non-limiting examples of liposomes can include, but are not limited to, the multilamellar vesicle (MLV), the small unilamellar liposome vesicle (SUV), the large unilamellar vesicle (LUV), the cochleate vesicle, and combinations thereof. In one example, the liposome can be substantially free of lecithin.

In another example, the lipid can form an inverted micelle which can include nanometer-sized (1-10 nm) water droplets dispersed in a non-polar solvent obtained by the action of surfactants. Non-limiting examples of surfactants can include, but are not limited to, AOT [sodium bis(2-ethyl hexyl) sulfosuccinate], CTAB (cetyltrimethylammonium bromide), dode-cyl penta(oxyethylene) ether (C12E5), n-dodecyloctaoxyethylene glycol monoether (C12E8), cetylbenzyldimethylammonium chloride (CBAC), didodecyl-dimethylammonium bromide (DDAB), sorbitan monooleate, and sodium dodecylbenzene-sulfonate (NaDBS), Triton X-100[polyoxyethylene(10)isooctylphenyl ether], poly-oxyethylene(4) lauryl ether (known as Brij30), pentaoxyethylene-glycol-nonyl-phenyl ether (known as Igepal-CO520), poly(oxyethylene)5nonylphenol ether (NP-5), poly(oxyethylene)9nonylphenol ether (NP-9), poly(oxyethylene)12 nonylphenol ether (NP-12), and combinations thereof.

In some examples, the support agent can include a preservative. Non-limiting examples of preservatives can include ascorbic acid, acetylcysteine, bisulfite, metabisulfite, monothioglycerol, phenol, meta-cresol, benzyl alcohol, methyl paraben, propyl paraben, butyl paraben, benzalkonium chloride, benzethonium chloride, butylated hydroxyl toluene, myristyl gamma-picolimium chloride, 2-phenoxyethanol, phenyl mercuric nitrate, chlorobutanol, thimerosal, tocopherols, the like, or combinations thereof.

In some examples, the support agent can include an adjuvant. Non-limiting examples of adjuvants can include, but are not limited to, analgesic adjuvants, aluminum hydroxide, aluminum phosphate, calcium phosphate hydroxide, paraffin oil, Bordetella pertussis, mycobacterium bovis, toxoids, squalene, detergents, plant saponins, cytokines, and combinations thereof.

In some examples, the support agent can include an emollient. Non-limiting examples of emollients can include, but are not limited to, aloe vera, lanolin, urea, petrolatum, shea butter, cocoa butter, mineral oil, paraffin, beeswax, squalene, jojoba oil, coconut oil, sesame oil, almond oil, cetyl alcohol, stearyl alcohol, olive oil, oleic acid, triethylhexanoin, glycerol, sorbitol, propylene glycol, cyclomethicone, dimethicone, the like, or combinations thereof.

The compositions and dosage forms described herein can include a variety of pharmaceutically acceptable carriers known in the art. At its most basic level, in some embodiments, a carrier can include water. In some embodiments, the water can be deoxygenated water. Additionally non-limited examples of components that can be included as components of the pharmaceutical carrier include lipophilic surfactants, hydrophilic surfactants, triglycerides, fatty acid, or fatty acid glycerides, and combinations thereof.

In some embodiments, the carrier of the composition can include a lipophilic additive. In some embodiments, the lipophilic additive can comprise at least about 50 wt% of the pharmaceutically acceptable carrier. Non-limiting examples of lipophilic additives can include lipophilic surfactants, triglycerides, tocopherol, tocopherol derivatives and combinations thereof. In one embodiment, the lipophilic additive can include a fatty acid or fatty acid glyceride. In another embodiment, lipophilic additive can include the fatty acid glyceride, and the fatty acid glyceride can be a monoglyceride, a diglyceride, or mixtures thereof. Non-limiting examples of fatty acid glycerides that can be used in the pharmaceutical compositions and dosage forms of the present disclosure include monoglycerides and/or diglycerides derived from sources such as maize oil, poppy seed oil, safflower oil, sunflower oil, borage seed oil, peppermint oil, coconut oil, palm kernel oil, castor oil, and mixtures thereof. In one embodiment, the pharmaceutical composition or dosage form thereof comprises 50% by weight or less of a triglyceride. In a specific embodiment, the pharmaceutical composition or dosage form thereof, comprises less than 50 % by weight of castor oil. In another embodiment, the composition includes 10 wt% or less of triglycerides. In a further embodiment, the composition includes 5 wt% or less of triglycerides. In a still a further embodiment, the composition includes about 3 wt% or less of triglycerides. In still a further embodiment, the composition includes about 1 wt% or less of triglycerides. In another embodiment, the composition is free or substantially free of triglycerides. In another embodiment, the composition and dosage forms are free of phytosterols and phytosterol fatty acid esters.

In another embodiment, the lipophilic additive can include a lipophilic surfactant. As used herein a surfactant is considered to be a lipophilic surfactant when it has an HLB value of 10 or less. Various lipophilic surfactants can be used including, but not limited to mono-, di- glycerides of fatty acids like glyceryl monolinoleate (e.g. Maisine® 35-1), mono- and di glycerides of caprylic, capric acid (e.g. Capmul® MCM), glyceryl monooleate, reaction mixtures of alcohols or polyalcohols with a variety of natural and/or hydrogenated oils such as PEG-5 hydrogenated castor oil, PEG-7 hydrogenated castor oil, PEG-9 hydrogenated castor oil, PEG-6 corn oil (e.g. Labrafil® M 2125 CS), PEG-6 almond oil (e.g. Labrafil®M 1966 CS), PEG-6 apricot kernel oil (e.g. Labrafil®M 1944 CS), PEG-6 olive oil (e.g.Labrafil®M 1980 CS), PEG-6 peanut oil (e.g. Labrafil®M 1969 CS), PEG-6 hydrogenated palm kernel oil (e.g. Labrafil®. M 2130 BS), PEG-6 palm kernel oil (e.g. Labrafil® M 2130 CS), PEG-6 triolein (e.g. Labrafil® M 2735 CS), PEG-8 corn oil (e.g. Labrafil® WL 2609 BS), PEG-20 corn glycerides (e.g. Crovol® M40), PEG-20 almond glycerides (e.g. Crovol® A40), lipophilic polyoxyethylene-polyoxypropylene block co-polymers (e.g. Pluronic® L92, L101, L121 etc.); propylene glycol fatty acid esters, such as propylene glycol monolaurate (e.g. Lauroglycol FCC), propylene glycol ricinoleate (e.g. Propymuls), propylene glycol monooleate (e.g. Myverol P-O6), propylene glycol dicaprylate/dicaprate (e.g. Captex® 200), and propylene glycol dioctanoate (e.g. Captex® 800), propylene glycol mono- caprylate (e.g. Capryol® 90); propylene glycol oleate (e.g. Lutrol OP2000); propylene glycol myristate; propylene glycol mono stearate; propylene glycol hydroxy stearate; propylene glycol ricinoleate ; propylene glycol isostearate; propylene glycol mono-oleate; propylene glycol dicaprylate/dicaprate; propylene glycol dioctanoate; propylene glycol caprylate-caprate; propylene glycol dilaurate; propylene glycol distearate; propylene glycol dicaprylate; propylene glycol dicaprate; mixtures of propylene glycol esters and glycerol esters such as mixtures composed of the oleic acid esters of propylene glycol and glycerol (e.g. Arlacel® 186); sterol and sterol derivatives such as cholesterol, sitosterol, phytosterol, phytosterol fatty acid esters, PEG-5 soya sterol, PEG-10 soya sterol, PEG-20 soya sterol, and the like; glyceryl palmitostearate, glyceryl stearate, glyceryl distearate, glyceryl monostearate, or a combination thereof; sorbitan fatty acid esters such as sorbitan monolaurate (e.g. Arlacel 20), sorbitan monopalmitate (e.g. Span-40), sorbitan monooleate (e.g. Span-80), sorbitan monostearate, and sorbitan tristearate, sorbitan monolaurate, sorbitan monopalmitate, sorbitan monooleate, sorbitan trioleate, sorbitan sesquioleate, sorbitan tristearate, sorbitan monoisostearate, sorbitan sesquistearate, and the like; fatty acids such as capric acid, caprylic acid, oleic acid, linoleic acid, myristic acid, menthol, menthol derivatives, lecithin, phosphatidyl choline, bile salts, and the like, and mixtures thereof. It can be noted that some lipophilic surfactants may also function as a solubilizer component of the compositions and dosage forms.

In one embodiment, the lipophilic surfactant can be selected from the group consisting of glyceryl monolinoleate (e.g. Maisine® 35-1), mono- and di glycerides of caprylic, capric acid (e.g. Capmul® MCM), glyceryl monooleate, propylene glycol mono caprylate, propylene glycol oleate, propylene glycol monostearate, propylene glycol monolaurate, propylene glycol mono-oleate, propylene glycol dicaprylate/dicaprate, sorbitan monooleate, PEG-5 hydrogenated castor oil, PEG-7 hydrogenated castor oil, PEG-9 hydrogenated castor oil, PEG-6 corn oil, PEG-6 almond oil, PEG-6 apricot kernel oil, PEG-6 olive oil, PEG-6 peanut oil, PEG-6 hydrogenated palm kernel oil, sorbitan monolaurate (e.g. Arlacel 20), sorbitan monopalmitate, sorbitan monooleate , sorbitan monostearate, sorbitan tristearate, sorbitan monolaurate, sorbitan monopalmitate, sorbitan monooleate, sorbitan trioleate, sorbitan sesquioleate, sorbitan tristearate, sorbitan monoisostearate, and combinations thereof. In some embodiments, the lipophilic surfactants can comprise at least about 50 wt% of the total pharmaceutically acceptable carrier. It should be noted that the combinations of two or more lipophilic surfactants from the same or different classes therein are also within the scope of this disclosure and are together can be referred to as the lipophilic surfactant, unless otherwise stated.

In embodiments of the present disclosure, the pharmaceutical compositions or dosage forms can include a hydrophilic additive. In one embodiment, hydrophilic additive is a selected from the group consisting of hydrophilic surfactant, celluloses - such as hydroxypropyl celluloses low molecular weight, low viscosity types (e.g. Methocel® E5, E6, E10 E15, LV100 etc. grades) and hydroxypropyl celluloses having higher molecular weight, medium to high viscosity (e.g. Methocel® K4M, K15M, K100M etc); polyvinylpyrrolidones (e.g. Kollidon k17, K30 etc); polyvinyl acetates and combinations thereof.

In one embodiment, the hydrophilic additive can be a hydrophilic surfactant. A surfactant is considered to be a hydrophilic surfactant when it has an HLB value of greater than 10. Non-limiting examples of hydrophilic surfactants include non-ionic surfactants, ionic surfactants and zwitterionic surfactants. Specifically the hydrophilic surfactants include, but are not limited to, alcohol-oil transesterification products; polyoxyethylene hydrogenated vegetable oils; polyoxyethylene vegetable oils; alkyl sulphate salts, dioctyl sulfosuccinate salts ; polyethylene glycol fatty acids esters; polyethylene glycol fatty acids mono- and di- ester mixtures; polysorbates, polyethylene glycol derivatives of tocopherol and the like. It should be noted that the combinations of two or more hydrophilic surfactants from the same or different classes are within the scope of this disclosure and are together can be referred to as the hydrophilic surfactant unless explicitly specified. In one embodiment, the hydrophilic additive can be a hydrophilic surfactant. Non-limiting examples of hydrophilic surfactants can include PEG-8 caprylic/capric glycerides, lauroyl macrogol-32 glyceride, stearoyl macrogol glyceride, PEG-40 hydrogenated castor oil, PEG-35 castor oil, sodium lauryl sulfate, sodium dioctyl sulfosuccinate, polyethylene glycol fatty acids mono- and di- ester mixtures, polysorbate 80, polysorbate 20, polyethylene glycol 1000 tocopherol succinate, phytosterols, phytosterol fatty acid esters, and mixtures thereof.

In some embodiments, surfactants utilized in the pharmaceutical compositions described herein include sterols and derivatives of sterols. In various embodiments, these surfactants are hydrophilic or lipophilic. Examples of hydrophilic sterol surfactants are lanosterol PEG-24 cholesterol ether (e.g. Solulan C-24, Amerchol), PEG-30 soya sterol (e.g. Nikkol BPS-30, from Nikko), PEG-25 phyto sterol (e.g. Nikkol BPSH-25 from Nikko), PEG-30 cholestanol (e.g. Nikkol DHC, from Nikko). Examples of Lipophilic Sterol Surfactants are Cholesterol, sitosterol, Phytosterol (e.g. GENEROL series from Henkel), PEG-5 soya sterol (e.g. Nikkol BPS-S, from Nikko), PEG-10 soya sterol (e.g. Nikkol BPS-10 from Nikko), PEG-20 soya sterol (e.g. Nikkol BPS-20 from Nikko).

In one embodiment, the hydrophilic surfactant can comprise at least about 20% of the total pharmaceutical carrier. In another embodiment, the hydrophilic surfactant can comprise at least about 5 wt% of the carrier. In another embodiment, the hydrophilic surfactant can comprise less than 5 wt% of the carrier.

In another embodiment, the compositions or the dosage forms of the current disclosure includes rGSH, wherein the rGSH comprises about 5 wt% to about 50 wt% of the composition or the dosage form, and wherein the composition includes about 50 wt% to about 100 wt% of lipophilic additive and 0 wt% to about 50 wt% of hydrophilic additive. In a specific embodiment, the lipophilic additive can be lipophilic surfactant and the hydrophilic additive can be hydrophilic surfactant. In one embodiment, the hydrophilic surfactant can comprise at least about 20% of the composition. In another embodiment, the hydrophilic surfactant can comprise at least about 5 wt% of the composition. In another embodiment, the hydrophilic surfactant can comprise less than 5 wt% of the composition.

In some embodiments, the pharmaceutical compositions or the dosage form can include both a lipophilic surfactant and hydrophilic surfactant. In one embodiment, the lipophilic surfactant and hydrophilic surfactant can be present in amounts such that the ratio of amount (wt%) of lipophilic surfactant to amount (wt%) of hydrophilic surfactant is greater than 2:1. In another embodiment, the lipophilic surfactant and hydrophilic surfactant can be present in amounts such that the ratio of amount (wt%) of lipophilic surfactant to amount (wt%) of hydrophilic surfactant is greater than 2.5:1. In another embodiment, the lipophilic surfactant and hydrophilic surfactant can be present in amounts such that the ratio of amount (wt%) of lipophilic surfactant to amount (wt%) of hydrophilic surfactant is greater than 3.5:1. In still another embodiment, the lipophilic surfactant and hydrophilic surfactant can be present in amounts such that the ratio of amount (wt%) of lipophilic surfactant to amount (wt%) of hydrophilic surfactant is at least 6.5:1.

In some embodiments, a stable reduced glutathione composition can comprise a combination of deoxygenated water and reduced glutathione (rGSH). In one example, the deoxygenated water has less than about 7 mg/L of oxygen. In one example, the deoxygenated water has less than about 5 mg/L of oxygen. In one example, the deoxygenated water has less than about 4 mg/L of oxygen. In one specific embodiment, the deoxygenated water has less than about 3 mg/L of oxygen. In another specific embodiment, the deoxygenated water has less than about 2 mg/L of oxygen. In another specific embodiment, the deoxygenated water has less than about 1 mg/L. In another specific embodiment, the deoxygenated water has less than about 0.5 mg/L of oxygen. In another specific embodiment, the deoxygenated water has less than about 0.1 mg/L of oxygen. Generally, the oxygen content of the water is referred to as “oxygen” and determined by known techniques such as oximetry.

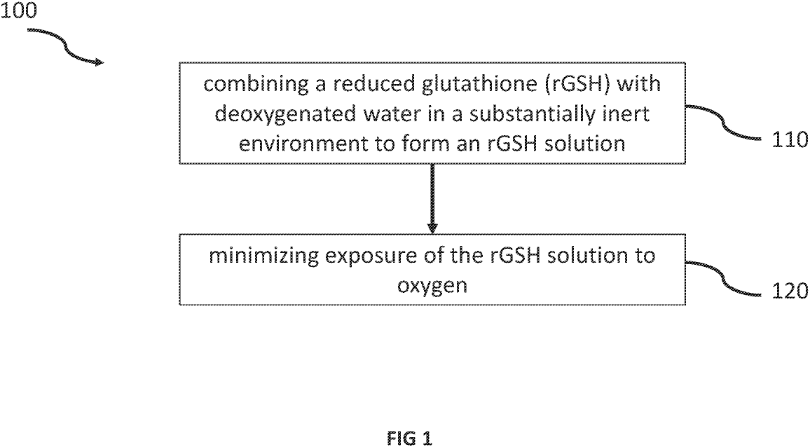

In some embodiments, as depicted in the flowchart in FIG. 1, a method of manufacturing 100 a stabilized reduced glutathione composition is provided. In one example, the method can comprise combining a reduced glutathione (rGSH) powder with deoxygenated water in a substantially inert environment to form an rGSH solution, as shown in block 110. In another example, the method can comprise minimizing exposure of the rGSH solution to oxygen, as shown in block 120. The inert environment can comprise an inert compound including one or more of nitrogen gas, helium gas, neon gas, argon gas, krypton gas, xenon gas, radon gas, a non-reactive compound, a non-oxidizing compound, the like, or combinations thereof.

In one example, an inert environment can be formed by blanketing the environment with an inert gas (e.g., nitrogen gas, N₂) to create an inert environment that is non-reactive and safe for mixing chemical compounds. Blanketing the environment with an inert gas (e.g., nitrogen gas) can prevent corrosion and oxidation. In one example, the inert environment can be an isolator under an inert gas atmosphere (e.g., a nitrogen gas atmosphere). In one example, the inert environment can have an oxygen concentration of less than one or more of: 5%, 2%, 1%, 0.5%, 0.1%, or combinations thereof.

In one example, deoxygenated water can be formed by removing dissolved O₂ gases from a water solvent. In one example, oxygen exposure of the rGSH solution can be minimized by continuously deoxygenating water by an inert gas bubbling, with the deoxygenated water and rGSH solution temperature maintained to avoid any reoxygenation. In one example, dissolved O₂ can be removed from water by one or more of: boiling water or reduced or atmospheric pressure, purging water with an inert gas, sonication of water under reduced pressure, mechanical means such as agitation and filtration, or gas removal based on differential pressure created through a duct, lumen, a sieve, or through a membrane, and the like.

In one example, the minimizing the exposure of the rGSH to oxygen can include minimizing an amount of oxygen in a container containing the rGSH solution. The amount of oxygen in a container containing the rGSH solution can be minimized in various ways. The container can be filled at room temperature (e.g., in the range of about 15° C. to about 30° C.). The operation can be performed at atmospheric pressure, i.e., without employing any substantial vacuum before, during, or after the filling process. In some aspects, the filling at atmospheric pressure can include an overlay or blanketing of the container of use with an inert gas such as nitrogen or argon before or after the rGSH solution has been introduced into the container. The container can be stoppered and sealed.

In one example, the container as used herein can be a container that is made of stainless steel or other suitable non-reactive material. Such containers may include glass-lined containers, polymeric bags, or other suitable containers that are generally employed in the compounding of pharmaceutical liquid preparations. In another example, the container can be a glass or plastic vial which contains the composition. Depending on the sterility restrictions, these vials may be further sterilized using heat, steam, gas, or radiation, among others.

In another embodiment, a vacuum can be used after the rGSH solution has been introduced into the container and the container has been blanketed with an inert gas, and the container is then stoppered and sealed. In a further example, a vacuum can be used after the rGSH solution has been introduced into the container and the container is sealed without blanketing with an inert gas, such as nitrogen or argon.

In one example, the method can further comprise combining the rGSH solution with a stabilizing agent. The stabilizing agent can be selected from a group consisting essentially of: phospholipids, glycolipids, cholesterol, triglycerides, fatty acids, fatty acid glycerides, surfactants, or a combination thereof.

In one example, the method can further comprise combining the rGSH solution with an additional active agent. The additional active agent can be a member selected from the group consisting of vitamin C, vitamin E, an antioxidant, N-acetyl-cysteine, hydroxytyrosol (HXT), superoxide dismutase (SOD), catalase, an anti-infective agent, an antibiotic, an anti-tumor agent, an anti-inflammatory agent, an analgesic, an anti-rheumatic agent, a supplementary growth factor, a supplementary cytokine, an amino acid, a protein, a vaccine, a hormone, a supplementary vitamin, oleuropein, and combinations thereof.

In one embodiment, a stable rGSH delivery system is provided. The system can comprise a container configured to minimize an amount of oxygen in the container. The system can further comprise a combination of rGSH and a substantially α-D-glucopyranoside-free stabilizing carrier within the container.

In one example, the container can be further configured to minimize the amount of oxygen in a headspace of the container. In another example, the headspace can be less than about 4% of the container volume. In another example, the headspace can be less than about 3% of the container volume. In another example, the headspace can be less than about 2% of the container volume. In another example, the headspace can be less than about 1% of the container volume. In another example, the amount of oxygen in the container can be less than about 5% of the container volume. In another example, the amount of oxygen in the container can be less than about 4% of the container volume. In another example, the amount of oxygen in the container can be less than about 3% of the container volume. In another example, the amount of oxygen in the container can be less than about 2% of the container volume. In one example, the container can include an inert compound including one or more of: nitrogen gas, helium gas, neon gas, argon gas, krypton gas, xenon gas, radon gas, a non-reactive compound, a non-oxidizing compound, the like, or combinations thereof.

In another example, the container can be a bag-on-valve container, a bag-in-bag container, a piston can container, or a can-in-can container, in which a bag can house the rGSH solution and can be attached directly to a valve or an internal can that can be inserted inside an external can.

In an example, the operations of production of a bag-on-valve, or can-in-can can comprise: (1) insert the bag on valve assembly, or the internal can, into the external can; (2) crimp the valve onto the external can; (3) fill the bag, or internal can, with the rGSH solution; (4) add a propellant into the space between the can and the bag, or internal can (during crimping or following crimping); (5) assemble an actuator suitable for dispensing the rGSH solution.

In an example, the operations for production of a bag-on-valve container, a bag-in-bag container, a piston can container, or a can-in-can container can vary based on their different mechanical structures, but the principles of assembling the system, filling it with the rGSH solution, and closing it can have similarities to the operations described in the preceding.

In an example, the container can be an inhaler device for respiratory therapy, which can facilitate treatment of organs of the respiratory system including: mucosal membranes, the oral cavity, the nasal cavity, the sinuses, the pharynx, the larynx, the trachea, the bronchus, and the lungs. In another example, the container can be an aerosol container.

### Formulations and Dosage Forms

The compositions and the dosage forms of the current disclosure can be formulated as one of a solution, a suspension, an emulsion, a gel, a hydrogel, a thermo-responsive gel, a cream, an ointment, a paste, an adhesive, an erodible matrix, a liquid reservoir, a patch, a powder, a compressed powder, or a combination thereof. The dosage forms of the current disclosure can comprise a transdermal dosage form, a parenteral dosage form, a topical dosage form, an oral dosage form, a nebulizer dosage form, a transmucosal dosage form, and combinations thereof.

The oral compositions of the present disclosure can be formulated to take any dosage form commonly known in the pharmaceutical arts such as solution, emulsion, granules, tablet or capsule. In one embodiment, the oral dosage form can be a capsule having a pharmaceutical composition of the present disclosure disposed therein. Both soft and hard gelatin and non-gelatin capsules can be used. The capsule size can be any size known in the art and can vary depending on the desired dosage amount. For instance, in one embodiment, the capsule can be a hard gelatin capsule having a fill volume of about 0.25 mL to about 1.1 mL. Similarly, in another embodiment, the capsule can be a soft gelatin capsule having a fill volume of about 0.25 mL to about 1.5 mL.

The compositions and the dosage forms (e.g. solution, emulsion, capsule or tablet) of the current disclosure can also include one or more of other additives selected from binders, bufferants, diluents, disintegrants, flavors, colorants, taste-masking agents, resins, pH modifiers, lubricants, glidants, thickening agent, opacifying agent, humectants, desiccants, effervescing agents, plasticizing agents and the like.

In a specific embodiment, the compositions of the current disclosure can be formulated in the form of granules, powder mixtures or tablets. In a specific embodiment, the rGSH present in the dosage form can be present in the form of nanoparticles or amorphous particles, or a mixture of both. In another specific embodiment, the rGSH present in these dosage form can be present in the form of crystalline, non-crystalline or amorphous particles or a mixtures thereof having an average particle size of about 2000 nm or less, 1500 nm or less, 1000 nm, 800 nm or less, 600 nm or less, 500 nm or less, 400 nm or less, 300 nm or less, 250 nm or less, 200 nm or less, 100 nm or less, 50 nm or less, or 25 nm or less; or the average particle size of said crystalline, non-crystalline or amorphous particles or a mixtures thereof is in the range 10 nm to 2000 nm, 10 nm to 1500 nm, 10 nm to 1000 nm, 10 nm to 800 nm, 10 nm to 750 nm; 10 nm to 600 nm, 10 nm to 500 nm, 10 nm to 400 nm, 10 nm to 300 nm, 10 nm to 250 nm, 10 nm to 200 nm, or 10 nm to 100 nm.

In another aspect of the disclosure, the oral pharmaceutical compositions and/or capsule dosage forms, namely the capsule fill, can include a solidifying agent. A solidifying agent is a pharmaceutically acceptable additive that is in a solid physical state at 20° C. Typically, solidifying agents facilitate the solidification of the pharmaceutical compositions of the present disclosure at temperatures around room temperature. The compositions and capsule fill of the present disclosure, including those with solidifying agents, can be non-liquid at standard temperature and pressure. In one embodiment, the composition and capsule fill can be semi-solid at standard temperature and pressure. In yet another embodiment, the composition and capsule fill can be solid at standard temperature and pressure. When present, the solidifying agent can comprise from about 0.1 wt% to about 25 wt% of the pharmaceutical composition or capsule dosage form. In another embodiment, the solidifying agent can comprise about 2 wt% to about 20 wt% of the composition or capsule dosage form. In yet a further embodiment, the solidifying agent can comprise about 3 wt% to about 15 wt% of the composition or capsule dosage form. In still a further embodiment, the solidifying agent can comprise about 3 wt% to about 9 wt% of the capsule fill. In yet a further embodiment, the solidifying agent can comprise 6 wt% to 9 wt% of the capsule fill. In one embodiment, the solidifying agent can melt at a temperature of about 45° C. to about 75° C. Non-limiting examples of solidifying agents that can be used include polyethylene glycols; sorbitol; gelatin; stearic acid; cetyl alcohol; cetosterayl alcohol; paraffin wax; polyvinyl alcohol; glyceryl stearates; glyceryl distearate; glyceryl monostearate; glyceryl palmitostearate; glyceryl behenate; waxes; hydrogenated castor oil; hydrogenated vegetable oil; bees wax, microcrystalline wax; sterols; phytosterols; phytosterols fatty acid esters, cholesterol and mixtures thereof. In one embodiment, the solidifying agent includes a polyethylene glycol (PEG) having molecular weight from about 1000 to about 20,000 and their mixtures. In another embodiment the solidifying agent includes one or more selected from the group consisting of polyethylene glycol; gelatin; stearic acid; polyvinyl alcohol; glyceryl stearates; glyceryl distearate; glyceryl monostearate; glyceryl palmitostearate; hydrogenated castor oil; hydrogenated vegetable oil and cholesterol.

In another specific embodiment, a solution of the rGSH can be in a carrier (e.g. lipophilic additive or hydrophilic additive or combinations thereof). Such solutions can be dispersed (e.g. by adsorption) in a solid carrier such colloidal silicon dioxide, lactose, calcium silicate, magnesium aluminum silicates, microcrystalline cellulose or combinations thereof, etc., and prepared as powder mixtures or granules or pellets to be disposed/filled into capsules or sachets, or admixed with tableting aids and compressed as tablets. Such sachets, capsules or tablets can also be formulated to contain an additional amount of the rGSH in crystalline and/or non-crystalline form, such that in the final composition or dosage form the total rGSH amount exists as a combination of at least two of the forms including solution, crystalline and non-crystalline forms, at about 20° C. or at about human body temperature or at 30° C. or above 30° C. including the range 30° C. to 40° C. In a further embodiment, these dosage forms provide serum rGSH levels and the pharmacokinetic parameters disclosed in the current disclosure for the rGSH upon single administration or two consecutive administrations or upon steady state.

In a further embodiment, the oral pharmaceutical composition can be formulated as dosage form to be administered to provide a daily rGSH dose of about 50 mg to about 1500 mg based on single unit or multiple unit dosing. In a specific embodiment, a single unit dosing comprises administering the entire required dose of the rGSH per administration time in the form of one unit dosage form; whereby the subject has to consume one unit dosage from per administration. In another specific embodiment, a multiple unit dosing comprises administering the entire required dose of the rGSH per administration time in the form of two or more unit dosage form; whereby the subject has to consume two, three, four or more unit dosages, per administration.

The dosage forms can be immediate release or controlled release (e.g. extended release, targeted release, pulsatile release, delayed release, etc.) or combinations thereof. When formulated as oral dosage forms, including the disclosed solution, capsule, or tablet dosage forms, the dosage forms can be formulated for once-a-day administration or for twice-a-day administration. The compositions and oral dosage forms can also be formulated for administration with a meal, including once-a-day administration with a meal. While the compositions dosage forms disclosed herein can be administered with a meal, a meal is not necessarily required.

In another example, rGSH can be present in the oral dosage form in an amount from about 50 mg to about 1500 mg. In another example, rGSH can be present in the oral dosage form in an amount from about 200 mg to about 1200 mg. In another example, rGSH can be present in the oral dosage form in an amount from about 300 mg to about 800 mg. In another example, rGSH can be present in the oral dosage form in an amount from about 400 mg to about 600 mg. In another example, rGSH can be present in the oral dosage form in an amount from about 300 mg to about 500 mg. In another example, rGSH can be present in the oral dosage form in an amount from about 600 mg to about 800 mg.

In another example, the stabilizing agent can be present in the oral dosage form in an amount from about 50 mg to about 1000 mg. In another example, the stabilizing agent can be present in the oral dosage form in an amount from about 100 mg to about 700 mg. In another example, the stabilizing agent can be present in the oral dosage form in an amount from about 200 mg to about 500 mg. In another example, the stabilizing agent can be present in the oral dosage form in an amount from about 300 mg to about 400 mg.

In another example, the parenteral dosage form can be suitable for administration via intradermal injection, subcutaneous injection, intramuscular injection, intraperitoneal injection, or intravenous injection.

The rGSH can be present in an amount of about 1 mg to about 1500 mg. The parenteral dosage forms can include a carrier comprising one or more of water, a tonicity agent, a buffering agent, a preservative, or a combination thereof. In another example, the rGSH can be present in an amount of about 50 mg to about 1500 mg. In another example, the rGSH can be present in an amount of about 5 mg to about 200 mg. In another example, the rGSH can be present in the composition in an amount of from about 10 mg to about 100 mg. In another example, the rGSH can be present in the composition in an amount of from about 25 mg to about 50 mg.

In some examples, the parenteral dosage form can include a tonicity agent. Non-limiting examples of tonicity agents can include sodium chloride, potassium chloride, calcium chloride, magnesium chloride, mannitol, sorbitol, dextrose, glycerin, propylene glycol, ethanol, trehalose, phosphate-buffered saline (PBS), Dulbecco’s PBS, Alsever’s solution, Tris-buffered saline (TBS), water, balanced salt solutions (BSS), such as Hank’s BSS, Earle’s BSS, Grey’s BSS, Puck’s BSS, Simm’s BSS, Tyrode’s BSS, and BSS Plus, the like, or combinations thereof. The tonicity agent can be used to provide an appropriate tonicity of the composition. In one aspect, the tonicity of the composition can be from about 250 to about 350 milliosmoles/liter (mOsm/L). In another aspect, the tonicity of the composition can be from about 277 to about 310 mOsm/L.

In some examples, the parenteral dosage form can include a pH adjuster or buffering agent. Non-limiting examples of pH adjusters or buffering agents can include a number of acids, bases, and combinations thereof, such as hydrochloric acid, phosphoric acid, citric acid, sodium hydroxide, potassium hydroxide, calcium hydroxide, acetate buffers, citrate buffers, tartrate buffers, phosphate buffers, triethanolamine (TRIS) buffers, the like, or combinations thereof. Typically, the pH of the therapeutic composition can be from about 5 to about 9, or from about 6 to about 8.

In another example, rGSH can be present in the parenteral dosage form in an amount from about 1 mg/mL to about 1500 mg/mL In another example, rGSH can be present in the parenteral dosage form in an amount from about 50 mg/mL to about 1500 mg/mL. In another example, rGSH can be present in the parenteral dosage form in an amount from about 5 mg/mL to about 100 mg/mL. In another example, rGSH can be present in the parenteral dosage form in an amount from about 10 mg/mL to about 50 mg/mL. In another example, rGSH can be present in the parenteral dosage form in an amount from about 15 mg/mL to about 30 mg/mL.

In another example, the stabilizing agent can be present in the parenteral dosage form in an amount from about 1 mg/mL to about 1200 mg/mL. In another example, the stabilizing agent can be present in the parenteral dosage form in an amount from about 50 mg/mL to about 1200 mg/mL. In another example, the stabilizing agent can be present in the parenteral dosage form in an amount from about 4 mg/mL to about 80 mg/mL. In another example, the stabilizing agent can be present in the parenteral dosage form in an amount from about 8 mg/mL to about 40 mg/mL. In another example, the stabilizing agent can be present in the parenteral dosage form in an amount from about 12 mg/mL to about 25 mg/mL.

The topical dosage forms can include a carrier comprising one or more of a solution, a gel, a cream, an ointment, a liniment, a paste, or a lotion. The rGSH can be present in an amount of about 50 mg to about 1500 mg. In another example, the rGSH can be present in an amount of about 200 mg to about 1200 mg. In another example, the rGSH can be present in an amount of about 300 mg to about 800 mg. In another example, the rGSH can be present in an amount of about 400 mg to about 600 mg.

The transdermal dosage forms can include a carrier including one or more of water, a polymer having surfactant properties, a polymer having thickening properties, or a solvent. The rGSH can be present in an amount of about 100 mg to about 1500 mg. In another example, the rGSH can be present in an amount of about 200 mg to about 1200 mg. In another example, the rGSH can be present in an amount of about 300 mg to about 800 mg. In another example, the rGSH can be present in an amount of about 400 mg to about 600 mg.

The transdermal dosage form can further comprise a penetration enhancer. The penetration enhancer can be present in an amount of about less than 5% by weight of composition. In another example, the penetration enhancer can be present in an amount of about less than 4% by weight of composition. In another example, the penetration enhancer can be present in an amount of about less than 3% by weight of composition. In another example, the penetration enhancer can be present in an amount of about less than 2% by weight of composition. In another example, the penetration enhancer can be present in an amount of about less than 1% by weight of composition.

In some examples, the transdermal dosage form can include a polymer having surfactant or emulsifying properties. Non-limiting examples of a polymer having surfactant or emulsifying properties can include, but are not limited to, hydrophobically modified polyacrylic acid commercially under the tradename PemulenTM TR-I and TR-2 by Lubrizol Corp., water-soluble or water-swellable copolymers based on acrylamidoalkyl sulfonic acid and cyclic N-vinylcarboxamides commercially available under the tradename Aristoflex® AVC by Clariant Corporation; water-soluble or water-swellable copolymers based on acrylamidoalkyl sulfonic acid and hydrophobically modified methacrylic acid commercially available under the tradename Aristoflex® HMB by Clariant Corporation and a homopolymer of acrylamidoalkyl sulfonic acid commercially available under the tradename Granthix APP by Grant Industries, Inc.

Other materials that may be suitable polymeric surfactants can include ethylene oxide/ propylene oxide block copolymers, sold under the trade name PLURONIC®, available from BASF Corporation of Parsippany, NJ., modified cellulose polymers such as those modified cellulose polymers described by the trade name KLUCEL®, available from Hercules Corporation of Wilmington, DE. Some examples include hydrophobically modified polyacrylic acid, acrylamidoalkyl sulfonic acid, cyclic N-vinylcarboxamides, acrylamidoalkyl sulfonic acid, hydrophobically modified methacrylic acid, a homopolymer of acrylamidoalkyl sulfonic acid, or combinations thereof as polymeric emulsifiers; and monomeric anionic surfactants, monomeric amphoteric surfactants, or combinations thereof as foaming agents. Some more examples are compositions that include hydrophobically modified polyacrylic acid; water-soluble or water-swellable copolymers based on acrylamidoalkyl sulfonic acid, cyclic N-vinylcarboxamides; water-soluble or water-swellable copolymers based on acrylamidoalkyl sulfonic acid, hydrophobically modified methacrylic acid; a homopolymer of acrylamidoalkyl sulfonic acid, or combinations thereof as polymeric emulsifiers, and include a betaine as the foaming surfactant. Some examples are compositions that include copolymers based on acrylamidoalkylsulfonic acids and cyclic N-vinylcarboxamides and/or linear N- vinylcarboxamides (e.g., Aristoflex® AVC and Aristoflex® HMB from Clariant Corporation) as polymeric emulsifiers and a betaine as foaming surfactant.

In some examples, the transdermal dosage form can include a polymer having thickening properties. Non-limiting examples of a polymer having thickening properties can include, but are not limited to, a hydrophobically modified cross-linked acrylate copolymer (Carbopol® Ultrez 20). Other polymers having similar properties may also be used. Non-limiting examples of polymers having thickening properties can include PEG-150 distearate, PEG-7 glyceryl cocoate, PEG-200 hydrogenated glyceryl palmitate, PEG-120 methyl glucose dioleate, carboxymethylene polymer, carboxyvinyl polymer, acrylates, C₁₀-C₃₀ alkyl acrylate crosspolymers, and combinations thereof.

The transdermal dosage form can further comprise a penetration enhancer. Non-limiting examples of a penetration enhancer can include, but are not limited to, ethanol, propylene glycol, oleic acid and other fatty acids, azone, terpenes, terpenoids, bile acids, isopropyl myristate and other fatty esters, dimethyl sulphoxides, N-methyl-2-pyrrolidone and other pyrrolidones, the like, or combinations thereof.

In another example, rGSH can be present in the transdermal dosage form in an amount from about 50 mg to about 1500 mg. In another example, rGSH can be present in the transdermal dosage form in an amount from about 200 mg to about 1200 mg. In another example, rGSH can be present in the transdermal dosage form in an amount from about 300 mg to about 800 mg. In another example, rGSH can be present in the transdermal dosage form in an amount from about 400 mg to about 600 mg.

In another example, the stabilizing agent can be present in the transdermal dosage form in an amount from about 50 mg to about 1000 mg. In another example, the stabilizing agent can be present in the transdermal dosage form in an amount from about 100 mg to about 700 mg. In another example, the stabilizing agent can be present in the transdermal dosage form in an amount from about 200 mg to about 500 mg. In another example, the stabilizing agent can be present in the transdermal dosage form in an amount from about 300 mg to about 400 mg.

The transmucosal dosage form can include rGSH in an amount of about 50 mg to about 1500 mg. The transmucosal dosage form can further include an adhesive layer, a bio-erodible matrix, or a solution. The transmucosal dosage form can be suitable for nasal, vaginal, or rectal administration.

In some examples, the transmucosal dosage form can include an adhesive, such as acrylic adhesives, polyisobutylene adhesives, silicon adhesives, hydrogel adhesives, the like, or combinations thereof.

In some examples, the transmucosal dosage form can include a bio-erodable matrix including, but are not limited to, biodegradable constituents, such as polylactic acid, poly(lactic-co-glycolic) acid, polyglycolic acid, poly(caprolactone), hyaluronic acid, polyhydroxybutyrate, polyvinyl alcohol, polyvinylpyrrolidone, carbomers, polyacrylic acid, polyoxyethylene/polyoxypropylene copolymers, other copolymers, albumins, casein, zein, collagen, other proteins, glucose, sucrose, maltose, trehalose, amylose, dextrose, fructose, mannose, galactose, other sugars, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, other sugar alcohols, chondroitin and/or other glycosaminoglycans, inulin, starches, acacia gum, agar, carboxymethyl cellulose, methyl cellulose, ethyl cellulose, alginates, carrageenan, cassia gums, cellulose gums, chitin, chitosan, curdlan, gelatin, dextran, fibrin, fulcelleran, gellan gum, ghatti gum, guar gum, tragacanth, karaya gum, locust bean gum, pectin, starch, tara gum, xanthan gum, and other polysaccharides, and functionalized derivatives of any of the above, copolymers thereof, the like, or mixtures thereof.

In another example, rGSH can be present in the transmucosal dosage form in an amount from about 50 mg to about 1500 mg. In another example, rGSH can be present in the transmucosal dosage form in an amount from about 200 mg to about 1200 mg. In another example, rGSH can be present in the transmucosal dosage form in an amount from about 300 mg to about 800 mg. In another example, rGSH can be present in the transmucosal dosage form in an amount from about 400 mg to about 600 mg.

In another example, the stabilizing agent can be present in the transmucosal dosage form in an amount from about 50 mg to about 1000 mg. In another example, the stabilizing agent can be present in the transmucosal dosage form in an amount from about 100 mg to about 700 mg. In another example, the stabilizing agent can be present in the transmucosal dosage form in an amount from about 200 mg to about 500 mg. In another example, the stabilizing agent can be present in the transmucosal dosage form in an amount from about 300 mg to about 400 mg.

Where the therapeutic composition is formulated for topical, transdermal, or transmucosal administration, the pharmaceutically acceptable carrier can include a variety of components suitable for forming a suspension, dispersion, lotion, cream, ointment, gel, foam, patch, powder, paste, sponge, the like, or a combination thereof. Non-limiting examples can include a solubilizer, an emulsifier, a dispersant, a thickener, an emollient, a pH adjuster, a tonicity agent, a preservative, an adhesive, a penetration enhancer, the like, or a combination thereof.

Non-limiting examples of solubilizers and/or emulsifiers can include water, ethanol, propylene glycol, ethylene glycol, glycerin, polyethylene glycol, banzalkonium chloride, benzethonium chloride, cetylpyridinium chloride, docusate sodium, nonoxynol-9, octoxynol, polyoxyethylene polyoxypropylene co-polymers, polyoxyl castor oils, polyoxyl hydrogenated castor oils, polyoxyl oleyl ethers, polyoxyl cetylstearyl ethers, polyoxyl stearates, polysorbates, sodium lauryl sulfate, sorbitan monolaurate, sorbitan monooleate, sorbitan monopalmitate, sorbitan monostearate, tyloxapol, the like, or combinations thereof.

In some examples, the solubilizer can also include a hydrocarbon or fatty substance, such as petrolatum, microcrystalline wax, paraffin wax, mineral oil, ceresi, coconut oil, bees wax, olive oil, lanolin, peanut oil, spermaceti wax, sesame oil, almond oil, hydrogenated castor oils, cotton seed oil, soybean oil, corn oil, hydrogenated sulfated castor oils, cetyl alcohol, stearyl alcohol, oleyl alcohol, lauryl alcohol, myristyl alcohol, stearic acid, oleic acid, palmitic acid, lauraic acid, ethyl oleate, isopropyl myristicate, the like, or combinations thereof.

In some examples, the solubilizer can include a silicon, such as polydimethylsiloxanes, methicones, dimethylpropylsiloxanes, methyl phenyl polysiloxanes, steryl esters of dimethyl polysiloxanes, ethoxylated dimethicones, ethoxylated methicones, the like, or combinations thereof.

In some additional examples, the therapeutic composition can include a dispersant and/or thickening agent, such as polyacrylic acids (e.g. Carbopols, for example), gelatin, pectin, tragacanth, methyl cellulose, hydroxylethylcellulose, hydroxypropylcellulose, HPMC, CMC, alginate, starch, polyvinyl alcohol, polyvinyl pyrrolidone, co-polymers of polyoxyethylene and polyoxypropylene, polyethylene glycol, the like, or combinations thereof.

The pH adjusters, tonicity agents, and preservatives in the topical, transdermal, or transmucosal therapeutic composition can generally include those pH adjusters and buffering agents, tonicity agents, and preservative agents listed above, or any other suitable pH adjusters, buffering agent, tonicity agent, or preservative for a particular formulation and/or use thereof. In some examples, the therapeutic composition can also include fumed silica, mica, talc, titanium dioxide, kaolin, aluminum glycinate, ethylenediaminetetraacetic acid, fragrances, colorants, other components as described above, the like, or combinations thereof.

In some additional examples, the pharmaceutically acceptable carrier can be formulated for administration via inhalation. In some examples, such formulations can include a propellant, such as hydrofluoralkanes, such as HFA134a, HFA227, or other suitable propellant. In yet other examples, the composition can be formulated for administration via nebulization. In either case, the composition can also include a variety of solubilizing agents, such as those described above. In other examples, the composition can be formulated as a dry powder aerosol. In some examples, the composition can include a particulate carrier and/or other particulate excipients, such as lactose, mannitol, other crystalline sugars, fumed silica, magnesium stearate, amino acids, the like, or combinations thereof.

In some specific examples, the pharmaceutically acceptable carrier can be formulated to provide a composition for ocular administration. Non-limiting examples can include topical application to the eye in the form of a drop, a gel, a film, an insert, a sponge, an ointment, the like, or a combination thereof. In yet other examples, the composition can be formulated for intraocular injection or implantation in the form of a solution, a depot, a scaffold, the like, or a combination thereof. Ocular compositions can include a variety of excipients, such as water, a tonicity agent, a thickening agent, a biodegradable polymer, a solubilizing agent, an emulsifier, a preservative, the like, or other suitable component, or a combination thereof.

It is noted that a number of compositional excipients are disclosed above with specific reference to particular types of formulations. However, it is noted that any excipients disclosed herein, or other suitable excipients, can be used with any type of therapeutic composition, where suitable, whether or not a particular excipient or type of excipient is specifically described in connection with that type of therapeutic composition. Therefore, the compositions described herein can be formulated in a variety of ways for various modes of administration.

In one embodiment, the composition or dosage form (e.g. capsule or tablet) can be administered with a meal, such as a meal that provides about 200 to about 1000 calories of energy of which 20-35% come from fats in the meal. In another embodiment, the composition or the dosage form can be administered with a standard meal. In another embodiment, the composition or capsule dosage form can be administered with a meal that provides about 50% of the calories derived from the fat. In another embodiment, the composition or the dosage form can be administered with a high-fat, high calorie meal. In another embodiment, the composition or the dosage form can be administered with a meal that provides about 500 to about 1000 calories of energy. In another embodiment, the composition or the dosage form can be administered with a meal that provides about 400 to about 700 calories derived from the fat therein. The compositional make-up of the meals that are administered can vary depending on the tastes and dietary needs of a subject. However, in some situations it may be beneficial to administer the compositions and dosage forms with meals that provide no fat or up to about 50 g of fat. In one embodiment, the meal can provide about 10 g to about 50 g of fat. In yet a further embodiment, the meal can provide about 20-35 g of fat.

In another embodiment, the composition or the dosage form can be administered orally with a meal that provides of the current disclosure can be administered to a subject, along with a meal such as breakfast, snack, food, lunch, dinner etc. In a specific embodiment, the meal can comprise about 15-55% fat. In another specific embodiment, the meal can comprise about 20-35% fat. In another specific embodiment, the meal can comprise about 20-55% fat. In another specific embodiment, the meal can comprise about 15-55% fat. In a specific embodiment, the compositions and the dosage forms containing rGSH of the current disclosure can enable to provide the said pharmacokinetic benefits to a subject when administered orally along with meal containing about 35 g ± 20 g fat content. In another embodiment, the serum pharmacokinetic benefit provided by the rGSH compositions and dosage forms of this disclosure when administered with a meal containing about 30% to 35% fat is not statistically significantly different compared that when administered with a meal containing as low as 15% to 20% fat or a meal containing as high as 50% to 55% fat.

In another embodiment, the oral pharmaceutical composition or dosage form can be formulated such that upon continuous once-a-day administration to each subject in a group of at least 4 subjects for a period of at least 28 days, 50% or less of the subjects in the group have a steady state serum rGSH concentration that falls below 55 µg/mL for more than 7 hours per day.

In another embodiment, the oral pharmaceutical composition or dosage form can be formulated such that upon continuous once-a-day administration to each subject in a group of at least 4 subjects for a period of at least 28 days, 25% or less of the subjects in the group have a steady state serum rGSH concentration that falls below 50 µg/mL for more than 7 hours per day.

In another embodiment, the oral pharmaceutical composition or dosage form can be formulated such that upon continuous twice daily administration to each subject in a group of at least 4 subjects for a period of at least 28 days, less than 50% of subjects in the group have a steady state serum rGSH concentration that falls below 55 µg/mL for more than 3.5 hours per day.

In another embodiment, the oral pharmaceutical composition or dosage form can be formulated such that upon continuous twice daily administration to each subject in a group of at least 4 subjects for a period of at least 28 days, 25% or less of subjects in the group have a steady state serum rGSH concentration that falls below 50 µg/mL for more than 3.5 hours per day.

In another embodiment, the oral pharmaceutical composition or dosage form (e.g. capsule or tablet) can be formulated such that upon continuous once or twice daily administration to each subject in a group of at least 4 subjects for a period of at least 28 days, the dosage form provides a steady state serum rGSH C_{avg} of 50 µg/mL to 100 µg/mL in at least 75% of the subjects in the group, and at least one of the following: 1) a serum GSSG Cₘₐₓ of less than 425 µg/mL in at least 75% of the subjects in the group; 2) a serum GSSG Cₘₐₓ of about 425 µg/mL to about 550 µg/mL in 25% or less of the subjects in the group; and 3) a serum GSSG Cₘₐₓ greater than 550 µg/mL in about 1% or less of the subjects in the group.

As discussed above, the present disclosure also provides for a method of treating a human subject in need of rGSH therapy is provided. The method can include administering any of the pharmaceutical compositions or dosage forms (e.g. capsule or tablet) disclosed herein. The pharmaceutical compositions and the dosage forms of the present disclosure can be used to treat any condition associated with rGSH deficiency. Examples of conditions associated with rGSH deficiency that can be treated using the dosage forms (e.g. capsule or tablet) and/or compositions of the present disclosure include, but are not limited to: psoriasis, eczema, acne, hives, warts, cold sores, candidiasis, athlete’s foot, wounds, mouth wounds, mouth pain, burns, sunburns, dry skin, wrinkles, blisters, actinic keratosis, rosacea, carbuncles, cellulitis, contact dermatitis, and keratosis pilaris, tardive dyskinesia, amyotrophic lateral sclerosis (ALS), primary lateral sclerosis (PLS), progressive muscular atrophy (PMA), progressive bulbar palsy, pseudobulbar palsy, monomelic amyotrophy (MMA), Bell’s palsy, cerebral palsy, multiple sclerosis (MS), muscular dystrophy, parkinson’s disease, and vertigoperipheral neuropathy, chemotherapy-induced peripheral neuropathy, diabetic peripheral neuropathy, concussion, neuralgia, copper deficiency myeloneuropathy, diabetic amyotrophy, hypothyroidism, paraneoplastic sensory neuronopathy, uremic neuropathy, carpal tunnel syndrome, herpes zoster, and median neuropathy, fibromyalgia, diabetes, arthritis, chronic obstructive pulmonary disease, rheumatoid arthritis, bronchitis, appendicitis, asthma, ulcers, tuberculosis, periodontitis, ulcerative colitis, Crohn’s disease, sinusitis, hepatitis, temporal arteritis, inflammatory bowel disease, allergies, shingles, post-herpetic neuralgia (PHN), HIV, herpes simplex encephalitis, chickenpox, measles, rubella, roseola, meningitis, encephalitis, warts, oral herpes, genital herpes, hepatitis, norovirus, rotavirus, adenovirus, flu, astrovirus, common cold, respiratory syncytial virus, parainfluenza, severe acute respiratory syndrome (SARS), middle east respiratory syndrome (MERS), coronavirus, and combinations, and combinations thereof.

Other conditions that can be treated by the compositions and dosage forms disclosed herein include acetaminophen overdose, drug-resistant bacterial infection, liver failure, HIV, herpetic encephalopathy, cystic fibrosis, narcotic overdose, macular degeneration, cataracts, glaucoma, peanut allergies, and combinations thereof.

In some embodiments, the rGSH compositions and the dosage forms thereof of the current disclosure can be used to treat or improve the symptoms of subjects suffering from conditions such as diabetic neuropathy, post-herpetic neuralgia (PHN), cold sore development, alcohol toxicity, paracetamol toxicity, wet macular degeneration, cataracts formation, corneal ulcers, , teeth cavities, sunburns, laser burns, Epstein-Barr virus diseases, wounds, scarring, chronic renal failure (CRF), nephropathy, heavy-metal poisoning, chemotherapy-induced aphthous mouth ulcers, post tonsil surgery, insect bites, poison ivy reactions, anaphylaxis, necrobiosis lipoidica diabeticorum, pulmonary fibrosis, chronic obstructive pulmonary disease (COPD), optic neuritis (ON), Leber hereditary optic neuropathy (LHON)-like optic neuropathy (LLON), non-arteritic anterior ischemic optic neuropathy (NAION), sporadic bilateral optic neuropathy (SBON), the like, and combinations thereof.

In some embodiments, the compositions and dosage forms of the rGSH of the current disclosure improves at least one of biological absorption and metabolic stability of the rGSH. In one embodiment, the biological absorption of the rGSH is intestinal lymphatic absorption.

In some embodiments, the compositions and dosage forms (e.g. capsule or tablets) of the current disclosure provides for a method of administering rGSH by finely adjusting the total rGSH equivalent dose administered such that various desired serum rGSH levels can be provided in individual subjects along with maintaining or controlling normal physiological levels of other active agents.

In another embodiment, the compositions or dosage of the current disclosure having rGSH can be administered for maintaining or controlling physiological levels of rGSH in a subject in need of rGSH therapy. In a further embodiment, the compositions or dosage of the current disclosure having rGSH can be administered in combination with other active agents for maintaining or controlling physiological levels of other active agents in a subject in need of rGSH therapy. In a further embodiment, the compositions or dosage of the current disclosure can have at least one of the immediate release, modified release, and targeted delivery properties in various regions of the GI tract and can be administered for maintaining or controlling physiological levels of rGSH in a subject in need of rGSH therapy.

Subjects that can be treated by the rGSH compositions and dosage form of the present disclosure can be any human subject in need thereof. In particular, in one embodiment, the human subject may be at least 14 years of age. In another embodiment, the human subject is an adult of at least age 30. In a further embodiment, the human subject can be at least age 50. In yet a further embodiment, the human subject can be an adult of at least age 60. In a further embodiment, the subject can be deficient in the endogenous rGSH levels.

### EXAMPLES

In one example, a stable reduced glutathione composition can comprise: a combination of deoxygenated water and reduced glutathione (rGSH) wherein more than about 80% of the glutathione remains in a reduced form after a period of about 365 days.

In one example, the rGSH can be present in the composition at a concentration of from about 5 wt% to about 20 wt%.

In one example, the deoxygenated water can have less than about 5 mg/L of oxygen.

In one example, the composition can further comprise an additional active agent.

In one example, the additional active agent can be a member selected from the group consisting of: an antioxidant, N-acetyl-cysteine, hydroxytyrosol (HXT), superoxide dismutase (SOD), catalase, an anti-infective agent, an antibiotic, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

In one example,_the composition can further comprise a support agent selected from the group consisting of: a stabilizing agent, a preservative, an adjuvant, an emollient, a carrier, sunflower oil, natural lemon essential oil, the like, and combinations thereof.

In one example, the amount of glutathione remaining in the reduced form after the period of 365 days can be greater than 90%.

In one example, the support agent can be a stabilizing agent.

In one example, the stabilizing agent can include an oxygen scavenger, a lipid, a synthetic polymer, a natural non-α-D-glucopyranoside polymer, a protein, an amino acid, a co-polymer, an inert gas, or a combination thereof.

In one example, the stabilizing agent can be a lipid selected from the group consisting essentially of: phospholipids, glycolipids, cholesterol, triglycerides, fatty acids, fatty acid glycerides, surfactants, or a combination thereof.

In one example, the lipid can form a liposome, an inverted micelle, or a combination thereof.

In one example, the composition can be formulated as one of a solution, a suspension, an emulsion, a gel, a hydrogel, a thermo-responsive gel, a cream, an ointment, a paste, an adhesive, an erodible matrix, a liquid reservoir, a patch, a powder, a compressed powder, or a combination thereof.

In one example, the composition can comprise a dosage form comprising a transdermal dosage form, a parenteral dosage form, a topical dosage form, an oral dosage form, a nebulizer dosage form, a transmucosal dosage form, and combinations thereof.

In one example, a therapeutically effective amount of the composition can provide a serum level of reduced glutathione in a subject that is between 5% and 50% greater than a baseline level for the subject during a period of time of up to 24 hours after administration.

In one example, the stable reduced glutathione composition can be substantially free of one or more of: α-D-glucopyranoside units; or macro-cyclic glucose subunits; or oligosaccharides; or glucopyranoside units; or glycosidic bonds.

In one example, a method of manufacturing a stabilized reduced glutathione composition can comprise: combining a reduced glutathione (rGSH) with deoxygenated water in a substantially inert environment to form an rGSH solution, and minimizing exposure of the rGSH solution to oxygen.

In one example, minimizing the exposure of the rGSH to oxygen can include minimizing an amount of oxygen in a headspace of a container containing the rGSH solution.

In one example, the method can comprise combining the reduced glutathione solution with a stabilizing agent.

In one example, the stabilizing agent can be a lipid selected from the group consisting essentially of: phospholipids, glycolipids, cholesterol, triglycerides, fatty acids, fatty acid glycerides, surfactants, or a combination thereof.

In one example, an additional active agent can be a member selected from the group consisting of vitamin C, vitamin E, an antioxidant, N-acetyl-cysteine, hydroxytyrosol (HXT), superoxide dismutase (SOD), catalase, an anti-infective agent, an antibiotic, an anti-tumor agent, an anti-inflammatory agent, , an analgesic, an anti-rheumatic agent, a supplementary growth factor, a supplementary cytokine, an amino acid, a protein, a vaccine, a hormone, a supplementary vitamin, oleuropein, and combinations thereof.

In one example, a stable reduced glutathione composition can comprise: an amount of reduced glutathione (rGSH) and a substantially α-D-glucopyranoside-free stabilizing carrier.

In one example, the stabilizing carrier can be deoxygenated water.

In one example, the deoxygenated water can have less than about 5 mg/L of oxygen.

In one example, the rGSH can be present in the composition at a concentration of from about 5 wt% to about 20 wt%.

In one example, the composition can further comprise an additional active agent.

In one example, the additional active agent can be a member selected from the group consisting of vitamin C, vitamin E, an antioxidant, N-acetyl-cysteine, hydroxytyrosol (HXT), superoxide dismutase (SOD), catalase, an anti-infective agent, an antibiotic, an anti-tumor agent, an anti-inflammatory agent, , an analgesic, an anti-rheumatic agent, a supplementary growth factor, a supplementary cytokine, an amino acid, a protein, a vaccine, a hormone, a supplementary vitamin, oleuropein, and combinations thereof.

In one example, the composition can further comprise a support agent that is a member selected from the group consisting of a stabilizing agent, a preservative, an adjuvant, an emollient, a carrier, sunflower oil, natural lemon essential oil, the like, and combinations thereof.

In one example, the amount of glutathione remaining in the reduced form after the period of 365 days can be greater than 80%.

In one example, the support agent can be a stabilizing agent.

In one example, the stabilizing agent can include an oxygen scavenger, a lipid, a synthetic polymer, a natural non-α-D-glucopyranoside polymer, a protein, an amino acid, a co-polymer, an inert gas, or a combination thereof.

In one example, the stabilizing agent can be a lipid selected from the group consisting essentially of: phospholipids, glycolipids, cholesterol, triglycerides, fatty acids, fatty acid glycerides, surfactants, or a combination thereof.

In one example, the lipid can form a liposome, an inverted micelle, or a combination thereof.

In one example, the composition can be formulated as one of a solution, a suspension, an emulsion, a gel, a hydrogel, a thermo-responsive gel, a cream, an ointment, an adhesive, an erodible matrix, a liquid reservoir, a patch, a powder, a compressed powder, or a combination thereof.

In one example, the composition can comprise a dosage form comprising a transdermal dosage form, a parenteral dosage form, a topical dosage form, an oral dosage form, a nebulizer dosage form, a transmucosal dosage form, and combinations thereof.

In one example, a therapeutically effective amount of the composition can result in a serum level of reduced glutathione in a subject that is between 5% and 50% greater than a baseline level for the subject during a period of time of up to 24 hours after administration.

In one example, the stable reduced glutathione composition can be substantially free of one or more of: macro-cyclic glucose subunits; or oligosaccharides; or glucopyranoside units; or glycosidic bonds.

In one example, a stable reduced glutathione delivery system can comprise: a container configured to minimize an amount of oxygen in the container; and a combination of reduced glutathione (rGSH) and a substantially α-D-glucopyranoside-free stabilizing carrier within the container.

In one example, the container can be further configured to minimize the amount of oxygen in a headspace of the container.

In one example, the headspace can be less than about 4% of the container volume.

In one example, the amount of oxygen in the container can be less than about 5% of the container volume.

In one example, the container can include an inert compound including one or more of: nitrogen gas, helium gas, neon gas, argon gas, krypton gas, xenon gas, radon gas, a non-reactive compound, a non-oxidizing compound, the like, or combinations thereof.

In one example, the container can be a bag-on-valve container, a bag-in-bag container, a piston can container, or a can-in-can container.

In one example, a method of treating a condition in a subject that is responsive to treatment with reduced glutathione can comprise: administering a therapeutically effective amount of the composition to the subject.

In one example, an oral dosage form for administration of a stable reduced glutathione (rGSH) can comprise: an amount of reduced glutathione (rGSH) and a substantially α-D-glucopyranoside-free stabilizing carrier which is suitable for oral administration.

In one example, the carrier can comprise one or more of: binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof.

In one example, the rGSH can be present in an amount of about 50 mg to about 1500 mg.

In one example, the oral dosage form can be a capsule or tablet.

In one example, the oral dosage form can be controlled release.

In one example, a topical dosage form for administration of a stable reduced glutathione (rGSH) can comprise: an amount of rGSH and a substantially α-D-glucopyranoside-free stabilizing carrier which is suitable for topical administration.

In one example, the carrier can be one or more of a gel, a cream, an ointment, a liniment, a paste, or a lotion.

In one example, the rGSH can be present in an amount of about 50 mg to about 1500 mg.

In one example, a transdermal dosage form for administration of a stable reduced glutathione (rGSH) can comprise an amount of rGSH and a substantially α-D-glucopyranoside-free stabilizing carrier which is suitable for transdermal administration.

In one example, the carrier can include one or more of water, a polymer having surfactant properties, a polymer having thickening properties, or a solvent.

In one example, the transdermal dosage form can further comprise a penetration enhancer.

In one example, the rGSH can be present in an amount of about 50 mg to about 1500 mg.

In one example, a parenteral dosage form for administration of a stable reduced glutathione (rGSH) can comprise: an amount of rGSH and a substantially α-D-glucopyranoside-free stabilizing carrier which is suitable for parenteral administration.

In one example, the carrier can comprise one or more of water, a tonicity agent, a buffering agent, a preservative, or a combination thereof.

In one example, the rGSH can be present in an amount of about 1 mg to about 1500 mg.

In one example, the dosage form can be suitable for administration via intradermal injection, subcutaneous injection, intramuscular injection, intraperitoneal injection, or intravenous injection.

In one example, a transmucosal dosage form for administration of a stable reduced glutathione (rGSH) can comprise: an amount of rGSH and a substantially α-D-glucopyranoside-free stabilizing carrier which is suitable for transmucosal administration.

In one example, the rGSH can be present in an amount of about 50 mg to about 1500 mg.

In one example, the transmucosal dosage form can further comprise one or more of an adhesive layer, a bio-erodible matrix, or a solution.

In one example, the dosage form can be suitable for nasal, vaginal, or rectal administration.

In one example, the stabilizing carrier can comprise deoxygenated water having less than about 5 mg/L of oxygen.

In one example, the dosage form can further comprise an additional active agent.

In one example, the dosage form can further comprise an additional active agent selected from the group consisting of vitamin C, vitamin E, an antioxidant, N-acetyl-cysteine, hydroxytyrosol (HXT), superoxide dismutase (SOD), catalase, an anti-infective agent, an antibiotic, an anti-tumor agent, an anti-inflammatory agent, , an analgesic, an anti-rheumatic agent, a supplementary growth factor, a supplementary cytokine, an amino acid, a protein, a vaccine, a hormone, a supplementary vitamin, oleuropein, and combinations thereof.

In one example, the dosage form can further comprise a support agent selected from the group consisting of a stabilizing agent, a preservative, an adjuvant, an emollient, a carrier, sunflower oil, natural lemon essential oil, the like, and combinations thereof.

In one example, the amount of glutathione remaining in the reduced form after the period of 365 days can be greater than 80%.

In one example, the dosage form can further comprise a stabilizing agent.

In one example, the dosage form can further comprise a stabilizing agent including an oxygen scavenger, a lipid, a synthetic polymer, a natural non-α-D-glucopyranoside polymer, a protein, an amino acid, a co-polymer, an inert gas, or a combination thereof.

In one example, the dosage form can further comprise a stabilizing agent that is a lipid selected from the group consisting essentially of: phospholipids, glycolipids, cholesterol, triglycerides, fatty acids, fatty acid glycerides, surfactants, or a combination thereof.

In one example, the dosage form can further comprise a lipid that forms a liposome, an inverted micelle, or a combination thereof.

In one example, a therapeutically effective amount of the dosage form can result in a serum level of reduced glutathione in a subject that is between 5% and 50% greater than a baseline level for the subject during a period of time of up to 24 hours after administration.

In one example, the dosage form can be substantially free of one or more of: α-D-glucopyranoside units; or macro-cyclic glucose subunits; or oligosaccharides; or glucopyranoside units; or glycosidic bonds.

### EXAMPLES

The following examples are provided to promote a more clear understanding of certain embodiments of the present disclosure, and are in no way meant as a limitation thereon.

### EXAMPLE 1 - rGSH Composition

An rGSH containing composition is prepared by using the components set forth in Table I. The composition is prepared by weighing all of the components into a clean stainless-steel container and combining with deoxygenated water in a substantially inert environment to form a solution. A predetermined quantity of the fill material is disposed into a capsule (for example, hard gelatin capsule) to get the required rGSH dose per dosage unit. The capsules are allowed to cool at room temperature, banded (if required) and packaged in a HDPE bottle and tightly closed with an appropriate lid.

TABLE I

| Example 1 | Composition mg/capsule |
|---|---|
| rGSH | 560 |
| Stabilizing agent | 420 |

### EXAMPLE 2 - rGSH Composition

An rGSH containing composition is prepared similarly to the method described in Example 1 using the components set forth in Table II.

TABLE II

| Example 2 | Composition mg/capsule |
|---|---|
| rGSH | 560 |
| Stabilizing agent | 420 |
| Solidifying agent (e.g. polyethylene glycol, 8000 or PEG 8000) | 40 |

### EXAMPLES 3 & 4 - rGSH Composition

rGSH containing composition are prepared similarly to the method described in Example 1 using the components set forth in Tables III and IV.

TABLE III

| Example 3 | Composition mg/capsule |
|---|---|
| rGSH | 320 |
| Stabilizing agent | 420 |

TABLE IV

| Example 4 | Composition mg/capsule |
|---|---|
| rGSH | 400 |
| Stabilizing agent | 420 |

### EXAMPLE 5 - rGSH Composition

An rGSH containing composition is prepared similarly to the method described in Example 1 using the components set forth in Table V.

TABLE V

| Example 5 | Composition mg/capsule |
|---|---|
| rGSH | 560 |
| Stabilizing agent | 150 |

### EXAMPLE 6 - rGSH Composition

An rGSH containing composition is prepared by using the components set forth in Table VI. The composition is prepared by weighing all of the components into a clean stainless-steel container and combining with deoxygenated water in a substantially inert environment to form a solution and encapsulated in a stabilizing agent. The composition was packaged and stored in a container.

TABLE VI

| Example 6 | Weight percent/Total Composition |
|---|---|
| rGSH | 14.0 |
| Stabilizing agent | 10.5 |

### EXAMPLE 7 - rGSH Composition

An rGSH composition is prepared by using the components set forth in Table VII and a method similar to that described in Example 6.

TABLE VII

| Example 7 | Weight percent/Total Composition |
|---|---|
| rGSH | 10.0 |
| Stabilizing agent | 10.5 |

### EXAMPLE 8 - rGSH Composition

An rGSH composition is prepared by using the components set forth in Table VIII and a method similar to that described in Example 6.

TABLE VIII

| Example 8 | Weight percent/Total Composition |
|---|---|
| rGSH | 8.0 |
| Stabilizing agent | 10.5 |

### EXAMPLE 9 - rGSH Composition

An rGSH composition is prepared by using the components set forth in Table IX and a method similar to that described in Example 6.

TABLE IX

| Example 9 | Weight percent/Total Composition |
|---|---|
| rGSH | 14.0 |
| Stabilizing agent | 4.0 |

### EXAMPLE 10 - rGSH Composition

An rGSH composition is prepared by using the components set forth in Table X and a method similar to that described in Example 6.

TABLE X

| Example 10 | Weight percent/Total Composition |
|---|---|
| rGSH | 14.0 |
| Stabilizing agent | 11.0 |

### EXAMPLE 11 - rGSH Composition

An rGSH composition is prepared using the components set forth in Table XI. Bubble water with an inert gas and reduce the dissolved oxygen content to 5 mg/L. Add the tonicity agent while stirring and continue stirring to get a clear solution. Add rGSH powder to the above solution under stirring. Add the stabilizing agent. Adjust the pH of the solution to 5.9 using 0.1 N NaOH/HCl. Make up the volume of the solution to the batch size with water for injection. The inert gas bubbling is continued throughout the solution preparation.

Filter the solution using PVDF filter, and fill the filtered aqueous composition into 100 mL glass vials at room temperature. Before stoppering the containers, layer inert gas over the aqueous composition to reduce the head space oxygen to less than 4% and seal them with appropriate seals. The sealed containers are sterilized in an autoclave at 121° C. for 30 minutes.

TABLE XI

| | | | | |
|---|---|---|---|---|
| Example 11 | Formulation 11a | Formulation 11b | Formulation 11c | Formulation 11d |
| rGSH | 14.0 mg/mL | 14.0 mg/mL | 14.0 mg/mL | 14.0 mg/mL |
| Stabilizing agent | 10.5 mg/mL | 10.5 mg/mL | 10.5 mg/mL | 10.5 mg/mL |
| Tonicity Agent (e.g., mannitol) | 20.0 mg/mL | 20.0 mg/mL | 20.0 mg/mL | 20.0 mg/mL |
| Water for injection | q.s. to 1 mL | q.s. to 1 mL | q.s. to 1 mL | q.s. to 1 mL |
| Sodium Hydroxide/HCl | q.s. to adjust pH 5.9 | q.s. to adjust pH 5.9 | q.s. to adjust pH 5.9 | q.s. to adjust pH 5.9 |

### EXAMPLES 12-15- rGSH Compositions

rGSH formulations for transdermal delivery of Examples 12-15 can include the components as set forth in Table XII. Bubble water with an inert gas and reduce the dissolved oxygen content to 5 mg/L. Emulsifiers are added while stirring water slowly until completely dissolved. Penetration enhancers are added and mixed thoroughly. Reduced glutathione powder can be added. The stabilizing agent can be added and allowed to mix for longer than 5 minutes. The resultant composition can be used immediately or can be stored in an environment substantially free of oxidation.

TABLE XII

| Components/ Attributes | Weight % | | | |
|---|---|---|---|---|
| | Example 12 | Example 13 | Example 14 | Example 15 |
| rGSH | 14 | 10 | 8 | 14 |
| Stabilizing Agent | 10.5 | 10.5 | 10.5 | 4 |
| Penetration Enhancer (e.g. Propylene Glycol) | 3% | 3% | 3% | 3% |
| Mineral oil | 1% | 1% | 1% | 1% |
| Emulsifier | 1% | 1% | 1% | 1% |
| pH adjuster | 0.05 | 0.05 | 0.05 | 0.05 |
| Water for injection | q.s. | q.s. | q.s. | q.s. |

It is understood that the above-described various types of compositions, dosage forms and/or modes of applications are only illustrative of preferred embodiments of the present disclosure. Numerous modifications and alternative arrangements may be devised by those skilled in the art without departing from the spirit and scope of the present disclosure and the appended claims are intended to cover such modifications and arrangements. Thus, while the present disclosure has been described above with particularity and detail in connection with what is presently deemed to be the most practical and preferred embodiments of the disclosure, it will be apparent to those of ordinary skill in the art that variations including, but not limited to, variations in size, materials, shape, form, function and manner of operation, assembly and use may be made without departing from the principles and concepts set forth herein.

1. A stable reduced glutathione composition, comprising:
a combination of deoxygenated water and reduced glutathione (rGSH) wherein more than about 80% of the glutathione remains in a reduced form after a period of about 365 days.

2. The composition of claim 1, wherein the rGSH is present in the composition at a concentration of from about 5 wt% to about 20 wt%.

3. The composition of claim 1, wherein the deoxygenated water has less than about 5 mg/L of oxygen.

4. The composition of claim 1, wherein the composition further comprises an additional active agent.

5. The composition of claim 4, wherein the additional active agent is a member selected from the group consisting of: an antioxidant, N-acetyl-cysteine, hydroxytyrosol (HXT), superoxide dismutase (SOD), catalase, an anti-infective agent, an antibiotic, an anti-tumor agent, an anti-inflammatory agent, an analgesic, an anti-rheumatic agent, a growth factor, a cytokine, an amino acid, a protein, a vaccine, a hormone, a vitamin, oleuropein, the like, and combinations thereof.

6. The composition of claim 1, wherein the composition further comprises a support agent selected from the group consisting of: a stabilizing agent, a preservative, an adjuvant, an emollient, a carrier, sunflower oil, natural lemon essential oil, the like, and combinations thereof.

7. The composition of claim 1, wherein the amount of glutathione remaining in the reduced form after the period of 365 days is greater than 90%.

8. The composition of claim 6, wherein the support agent is a stabilizing agent.

9. The composition of claim 8, wherein the stabilizing agent includes an oxygen scavenger, a lipid, a synthetic polymer, a natural non-a-D-glucopyranoside polymer, a protein, an amino acid, a co-polymer, an inert gas, or a combination thereof.

10. The composition of claim 8, wherein the stabilizing agent is a lipid selected from the group consisting essentially of: phospholipids, glycolipids, cholesterol, triglycerides, fatty acids, fatty acid glycerides, surfactants, or a combination thereof.

11. The composition of claim 10, wherein the lipid forms a liposome, an inverted micelle, or a combination thereof.

12. The composition of claim 1, wherein the composition is formulated as one of a solution, a suspension, an emulsion, a gel, a hydrogel, a thermo-responsive gel, a cream, an ointment, a paste, an adhesive, an erodible matrix, a liquid reservoir, a patch, a powder, a compressed powder, or a combination thereof.

13. The composition of claim 1, wherein the composition comprises a dosage form comprising a transdermal dosage form, a parenteral dosage form, a topical dosage form, an oral dosage form, a nebulizer dosage form, a transmucosal dosage form, and combinations thereof.

14. The composition of claim 1, wherein a therapeutically effective amount of the composition provides a serum level of reduced glutathione in a subject that is between 5% and 50% greater than a baseline level for the subject during a period of time of up to 24 hours after administration.

15. The composition of claim 1, wherein the stable reduced glutathione composition is substantially free of one or more of:
a-D-glucopyranoside units; or
macro-cyclic glucose subunits; or
oligosaccharides; or
glucopyranoside units; or
glycosidic bonds.

16-55. (canceled)

56. A parenteral dosage form for administration of a stable reduced glutathione (rGSH), comprising:
an amount of rGSH and a substantially a-D-glucopyranoside-free stabilizing carrier which is suitable for parenteral administration.

57. The parenteral dosage form of claim 56, wherein the carrier comprises one or more of water, a tonicity agent, a buffering agent, a preservative, or a combination thereof.

58. The parenteral dosage form of claim 56, wherein the rGSH is present in an amount of about 1 mg to about 1500 mg.

59. The parenteral dosage form of claim 56, wherein the dosage form is suitable for administration via intradermal injection, subcutaneous injection, intramuscular injection, intraperitoneal injection, or intravenous injection.

60-67. (canceled)

68. The dosage form as recited in any one of claim 56, and wherein the amount of glutathione remaining in the reduced form after the period of 365 days is greater than 80%.

69-74. (canceled)
